(19) United States
(12) Patent Application Publication
(10) Pub. No.: US 2025/0090854 A1
(43) Pub. Date: Mar. 20, 2025

(54) ALZHEIMER’S DISEASE PREVENTION AND TREATMENT WITH LOW INTENSITY MAGNETIC STIMULATION APPLIED AT PHYSIOLOGICALLY RELEVANT FREQUENCIES

(71) Applicant: Actipulse Neuroscience, Inc, Cambridge, MA (US)

(72) Inventors: Alain Châtillon, Mexico City (MX); Gabriel Villafuerte, Mexico City (MX); Adrien Châtillon, Mexico City (MX)

(21) Appl. No.: 18/468,939

(22) Filed: Sep. 18, 2023

(51) Int. Cl. A61N 2/00; A61B 5/00; A61N 2/02

(52) U.S. Cl. CPC A61N 2/006; A61B 5/4088; A61N 2/02

(57) ABSTRACT

The present disclosure provides, in part, methods of non-invasive brain stimulation, e.g., repetitive magnetic stimulation (rTMS/gTMS), for treating and/or preventing mild cognitive impairment (MCI), dementia, and/or Alzheimer's Disease (AD), as well as progression from MCI to AD and/or dementia.

### FIELD

The present disclosure relates to, in part, clinical applications of non-invasive brain stimulation by repetitive transcranial magnetic stimulation (rTMS/gTMS) for the treatment and/or prevention of neurological disorders and diseases, e.g., Alzheimer's disease.

### BACKGROUND

Alzheimer's disease (AD) is a progressive neurodegenerative disorder that is estimated to be the cause of 60% or more of the cases of dementia. AD manifests as a range of clinical symptoms, including memory and cognitive issues, such as forgetfulness, difficulty with language, disorientation, mood swings and self-neglect. As AD progresses it can result in an accumulation of cellular damage in the brain accompanying increasing cognitive deficits, followed by eventual death. AD is associated with a condition broadly referred to as “mild cognitive impairment” (MCI), which is a progressive disease that is diagnosed when loss of memory and critical thinking skills are beyond what would be expected for an individual, given their age and educational background, where the loss is not so severe as to merit a diagnosis of dementia. MCI is thought to be a transitional state between normal aging and AD. The conversion rate from MCI to AD is estimated to be approximately 10% per year, increasing annually.

Mechanistically, the onset of MCI and its eventual progression to AD is thought to be caused by an aberrant buildup of amyloid beta (Aβ), accumulating extracellularly as amyloid plaques and/or tau plaques, and/or intracellularly as neurofibrillary tangles. These proteinaceous inclusions form in the brain, disrupting neuronal cell calcium homeostasis, interrupting glucose metabolism, broadly inhibiting housekeeping and catabolic enzymes, and eventually inducing apoptosis. The cell death disrupts neuronal functioning and connectivity, resulting in a progressive loss of brain function. There is an age-related loss in the ability to degrade or otherwise clear the proteins and/or inclusions, which creates a feedback loop where once damage beings, it can quickly accumulate leading to a cascade of inflammatory cytokines and alterations in the distribution of neurotrophic factors that would respond to damage.

Currently, there are no approved treatments proven to substantially stop and/or reverse the neural damage that causes MCI and/or its progression to AD. Pharmacological mitigation and treatment strategies have focused on improving the cognitive/behavioral aspects, for example using small molecules such as acetylcholinesterase inhibitors (e.g., tacrine, rivastigmine, galantamine, and donepezil), NMDA receptor antagonists (e.g., memantine); gene therapy (AAVs) to replace defect or aggregate-prone forms; vaccine-based immunotherapy (Aβ encoding, or nucleic acid encoding the peptides) to bolster immune clearance of aggregates; antibody-based treatments (e.g., bapineuzumab, solanezumab, crenezumab, BAN₂₄₀₁, aducanumab, and ALZ801) to solubilize plaques; and liposome, micelle, and metal nanoparticle-based drug delivery formulations for tissue targeting. However, each of these treatment modalities suffers from a variety of drawbacks, such as unfavorable clearance kinetics, off-target effects, poor tissue penetrance, and difficulty traversing the blood-brain barrier (BBB).

As an alternative and/or adjunctive therapy in addressing the shortcomings of pharmacological intervention, transcranial magnetic stimulation (TMS) is a non-invasive, painless neurostimulation technique with high safety profile that has been used as a neurologic and psychiatric treatment. However, the use of TMS as a treatment for neurocognitive diseases and disorders is expensive, requires a clinical setting, is hard to transport, complex to use, often requires external cooling, and could potentially trigger adverse effects if not carefully tuned, such as seizures. These disadvantages are the consequence of current TMS technology, which uses magnetic pulse intensities near 1 Tesla (e.g., 1,000,000 milligauss, or 10,000 gauss), but is poorly developed for improving patient outcomes.

There remains a need for devices and methods that employ non-invasive brain stimulation, e.g., TMS, with more precise parameters and implementation and without using large magnetic fields, unwieldy equipment, and without laborious focusing of the magnetic waves at certain points in the brain. There is a further need for a user-friendly device that is portable, appropriate for home use, and is safe to use without the need of a medical professional for TMS application.

### SUMMARY

Accordingly, the present disclosure provides, in part, a repetitive transcranial magnetic stimulation (rTMS/gTMS) method for treating or preventing Alzheimer's disease, comprising repetitively applying a magnetic pulse to the scalp of a subject in need thereof thereby stimulating neurons in the brain of the subject, wherein the magnetic pulse is applied repetitively over the subject's brain and at a frequency of less than about 50 Hz and an intensity of about 20,000 milligauss (20 gauss) to about 250,000 milligauss (250 gauss).

In aspects, the present disclosure provides, in part, a repetitive transcranial magnetic stimulation (rTMS/gTMS) method slowing or preventing a conversion of mild cognitive impairment (MCI) to Alzheimer's disease, comprising repetitively applying a magnetic pulse to the scalp of a subject in need thereof thereby stimulating neurons in the brain of the subject, wherein the magnetic pulse is applied repetitively over the subject's brain and at a frequency of less than about 50 Hz and an intensity of about 20,000 milligauss (20 gauss) to about 250,000 milligauss (250 gauss).

In embodiments, the magnetic pulse is applied at a frequency of about 15 Hz to about 30 Hz or about 30 Hz to about 40 Hz. In embodiments, the magnetic pulse is applied at a frequency of about 40 Hz.

In embodiments, the magnetic pulse is applied at an intensity of 20,000 to about 25,000 milligauss, about 25,000 to about 30,000 milligauss, about 30,000 to about 35,000 milligauss, about 35,000 to about 40,000 milligauss, about 45,000 to about 50,000 milligauss, about 55,000 to about 60,000 milligauss, about 65,000 to about 70,000 milligauss, about 75,000 to about 80,000 milligauss, about 85,000 to about 90,000 milligauss, about 95,000 to about 100,000 milligauss, about 20,000 to about 100,000 milligauss, about 100,000 to about 120,000 milligauss, about 120,000 to about 140,000 milligauss, about 140,000 to about 160,000 milligauss, about 160,000 to about 180,000 milligauss, about 180,000 to about 200,000 milligauss, about 100,000 to about 200,000 milligauss, or about 200,000 to about 250,000 milligauss. In embodiments, the magnetic pulse is applied at an intensity of about 100,000 milligauss (100 gauss).

In embodiments, the magnetic pulse generates an electric field of about 0.1 V/m² to about 10 V/m². In embodiments, the magnetic pulse generates an electric field of about 0.5 V/m² to about 1.5 V/m². In embodiments, the magnetic pulse generates an electric field of about 1 V/m².

In embodiments, the method is performed at least once daily, twice daily, thrice daily, or at least four times daily. In embodiments, the method is performed twice daily.

In embodiments, the method is performed for greater than about 15 minutes. In embodiments, wherein the method is performed for about 15 to about 60 minutes. In embodiments, the method is performed for about 30 minutes.

In embodiments, the one or more magnetic pulses are applied at least about 100 to at least about 500 times. In embodiments, the one or more magnetic pulses are applied at least about 100 to at least about 150 times. In embodiments, the one or more magnetic pulses are applied about at least 110 times. In embodiments, the one or more magnetic pulses are applied discontinuously. In embodiments, the one or more magnetic pulses last for a period of at least about 2 seconds, at least about 4 seconds, at least about 6 seconds, at least about 8 seconds, at least about 10 seconds, at least about 12 seconds, at least about 14 seconds, at least about 16 seconds, at least about 18 seconds, or at least about 20 seconds. In embodiments, the one or more magnetic pulses last for a period of at least about 10 seconds. In embodiments, the one or more magnetic pulses last for a period of about 10 seconds followed by a period of at least about 1 second without a magnetic pulse.

In embodiments, the method is applied to the subject chronically. In embodiments, the method is applied for at least about 2 months. In embodiments, the method is applied for at least about 6 months. In embodiments, the method is applied for at least about one year.

In embodiments, the method is self-applied.

In embodiments, the one or more magnetic pulses are applied using a device configured for conducting an electric current through one or more coils. In embodiments, the device generates a magnetic field. In embodiments, the device is suitable for home use. In embodiments, the device is portable. In embodiments, the device is wearable. In embodiments, the device is configured for use as a helmet or headband.

In embodiments, the subject is afflicted with MCI. In embodiments, the subject is afflicted with amnestic type MCI. In embodiments, the subject is afflicted with non-amnestic type MCI.

In embodiments, the subject is afflicted with preclinical Alzheimer's disease. In embodiments, the subject is afflicted with mild Alzheimer's disease. In embodiments, the subject is afflicted with moderate Alzheimer's disease. In embodiments, the subject is afflicted with mild dementia.

In embodiments, methods herein further comprise measuring and/or assessing at least one biomarker indicative of AD, selected from high amyloid beta (Aβ) in cerebrospinal fluid, high Tau in cerebrospinal fluid, and the presence of the ApoE4 allele. In embodiments, the subject presents as having at least one biomarker indicative of AD, selected from high amyloid beta (Aβ) in cerebrospinal fluid, high Tau in cerebrospinal fluid, and the presence of the ApoE4 allele.

In embodiments, methods herein further comprise measuring and/or assessing one or more symptoms of the AD and/or MCI, wherein the measuring and/or assessing occurs prior to applying the rTMS/gTMS and/or after applying the rTMS/gTMS. In embodiments, the measuring and/or assessing is performed using one or more methods, tests, and/or scales listed in Table 1.

In embodiments, the measuring and/or assessing is performed using Face-Name Associative Memory Exam (F-NAME) test, Rey Auditory Verbal Learning Test (RAVLT), Phonetic Verbal Fluency Test (pVFT), Semantic Verbal Fluency Test (SVFT), Montreal Cognitive Assessment (MoCA), Alzheimer's Disease Assessment Scale-Cognitive Subscale (ADAS-Cog), Clinical Dementia Rating (CDR), and/or a Clinical Dementia Rating Scale-Sum of Boxes (CDR-SB).

In embodiments, the subject presents as having a test score of one or more identified instruments for assessment of AD and/or MCI symptoms that is considered to indicate the presence of cognitive impairment or AD. In embodiments, the one or more identified instruments for assessment of AD and/or MCI symptoms is a Face-Name Associative Memory Exam (F-NAME).

In embodiments, the subject present as having a F-NAME test score of less than about 25.

In embodiments, the one or more identified instruments for assessment of AD and/or MCI symptoms is a Montreal Cognitive Assessment (MoCA). In embodiments, the subject present as having a MoCA test score of less than about 26.

In embodiments, the one or more identified instruments for assessment of AD and/or MCI symptoms is an Alzheimer's Disease Assessment Scale-Cognitive Subscale (ADAS-Cog). In embodiments, the subject presents as having a ADAS-Cog test score of 2, 3, 4, or 5 on one or more subscores of the test.

In embodiments, the one or more identified instruments for assessment of AD and/or MCI symptoms is a Clinical Dementia Rating (CDR) and/or a Clinical Dementia Rating Scale-Sum of Boxes (CDR-SB). In embodiments, the subject presents as having a CDR score and/or CDR-SB score of at least about 0.5, at least about or 1, or at least about 2.

In embodiments, the method stimulates neurons around about 2 to about 3 cm from the skull. In embodiments, the method stimulates alterations in gamma, beta, alpha, and/or theta oscillation in the subject's brain. In embodiments, the method stimulates alterations in gamma oscillation in the subject's brain. In embodiments, the method stimulates alterations in theta oscillation in the subject's brain.

In embodiments, the method substantially stimulates neurons throughout the subject's brain. In embodiments, the method substantially stimulates neurons in one or more of the cerebral cortex of the brain, left hemisphere of the subject's brain, left prefrontal dorsolateral cortex, and/or frontal lobe of the subject's brain. In embodiments, the method substantially stimulates neurons in the cerebral cortex of the subject. In embodiments, the method stimulates neurons outside of the subject's left prefrontal dorsolateral cortex.

In embodiments, the method prevents and/or delays the progression of MCI to Alzheimer's disease.

In embodiments, the method improves cognitive traits of the subject. In embodiments, the method prevents diminution of cognitive traits of the subject. In embodiments, the method slows the subject's memory loss and/or retains and/or increases memory capacity, memory function, and/or cognitive function in the subject.

In embodiments, memory capacity, memory function, cognitive function, and/or memory loss is assessed using one or more of the Instruments for assessment of AD symptoms listed in Table 1. In embodiments, an increase in one or more of a F-NAME score, a RAVLT score, a pVFT score, and a sVFT score following administration of the rTMS/gTMS indicates one or more of increased memory capacity, memory function, or cognitive function in the subject. In embodiments, an increase in a MoCA score and/or a decrease in an ADS-Cog score following administration of the rTMS/gTMS indicates one or more of increased memory capacity, memory function, or cognitive function in the subject. In embodiments, the method increases the subject's MoCA score, as compared to a MoCA score at baseline. In embodiments, the treatment decreases the subject's ADAS-COG score, as compared to an ADAS-COG score at baseline.

In embodiments, the method is substantially free of adverse effects, optionally selected from epileptic seizures, nausea, headache, vagal response, musculoskeletal pain, scalp petechial rash, scalp pain, edema, and bruising.

In embodiments, the method further comprises administering one or more treatments as a function of the subject's response to applying the rTMS/gTMS.

In embodiments, the subject's response to the rTMS/gTMS indicates a change in one or more symptoms of the AD, MCI, and/or dementia, and wherein the change informs the administering of the one or more treatments. In embodiments, the subject's response to the rTMS/gTMS is measured and/or assessed by one or more methods, tests, and/or scales listed in Table 1. In embodiments, the one or more treatments is administered prior to applying the rTMS/gTMS, substantially simultaneously with applying the rTMS/gTMS, or after applying the rTMS/gTMS. In embodiments, the one or more treatments is for AD, MCI, and/or dementia.

In embodiments, the one or more treatments comprises a pharmacological treatment, psychosocial treatment, caregiving treatment, diet-based treatment, and/or exercise.

In embodiments, the pharmacological treatment comprises one or more of neurological drugs, corticosteroids, antibiotics, antiviral agents, anti-Tau antibodies, Tau inhibitors, anti-amyloid beta antibodies, beta-amyloid aggregation inhibitors, anti-BACE1 antibodies, BACE1 inhibitors, acetylcholinesterase inhibitors (tacrine, rivastigmine, galantamine, and donepezil), NMDA receptor antagonists (memantine), gene therapies (AAVs), vaccine-based immunotherapy agents (AB peptide and/or nucleic acid encoding AB peptide), antibody-based agents (bapineuzumab, solanezumab, crenezumab, BAN2401, aducanumab, ALZ801); donepezil (ARICEPT), rivastigmine (EXELON), galantamine (RAZADYNE), and memantine (NAMENDA); and/or a liposome, micelle, and/or metal nanoparticle-based drug delivery agent.

In embodiments, the psychosocial treatment comprises speech therapy, behavioral therapy, cognitive therapy, cognitive/memory training, reminiscence therapy, validation therapy, supportive psychotherapy, sensory integration (Snoezelen), simulated presence therapy, and/or pet therapy.

In embodiments, the caregiving therapy comprises hospice care, feeding tubes, safety locks, and/or movement aids.

In embodiments, the diet-based treatment comprises liquid diets, pureed foods, and/or diets for difficulty swallowing.

In embodiments, the method obviates the need for one or more treatments.

In embodiments, the method further comprises administering one or more additional therapeutic agents. In embodiments, the one or more additional therapeutic agents is administered prior to applying the rTMS/gTMS, substantially simultaneously with applying the rTMS/gTMS, or after applying the rTMS/gTMS. In embodiments, the one or more additional therapeutic agents comprises one or more therapeutic agents for AD, MCI, and/or dementia. In embodiments, the one or more therapeutic agents for AD, MCI, and/or dementia comprises a neurological drug, corticosteroid, antibiotic, antiviral agent, anti-Tau antibody, Tau inhibitor, anti-amyloid beta antibody, beta-amyloid aggregation inhibitor, anti-BACE1 antibody, BACE1 inhibitor, acetylcholinesterase inhibitor (tacrine, rivastigmine, galantamine, and donepezil), NMDA receptor antagonist (memantine), gene therapy (AAVs), vaccine-based immunotherapy agent (AB peptide and/or nucleic acid encoding Aβ peptide), antibody-based agent (bapineuzumab, solanezumab, crenezumab, BAN2401, aducanumab, ALZ801); donepezil (ARICEPT), rivastigmine (EXELON), galantamine (RAZADYNE), and memantine (NAMENDA); and/or a liposome, micelle, and/or metal nanoparticle-based drug delivery agent.

### DESCRIPTION OF THE DRAWINGS

FIG. 1 depicts a non-limiting diagrammatic representation of a first part of a rTMS/gTMS device showing a housing component.

FIG. 2 depicts a non-limiting diagrammatic representation of a second part of a rTMS/gTMS device showing a magnetic coil arrangement.

FIG. 3 depicts a non-limiting diagrammatic representation of a third part of a rTMS/gTMS device as affixed to a subject's head.

FIG. 4 depicts a diagrammatic representation of a randomized, double-blind, sham-controlled, crossover study. The study included two sessions with a one-week interval therebetween. In the first session, participants were randomly assigned to either the sham or gTMS stimulation group. The session commenced with a comprehensive baseline cognitive evaluation, consisting of the Rey Auditory Verbal Learning Test (RAVLT), semantic Verbal Fluency Test (sVFT), and phonetic Verbal Fluency Test (pVFT) tests as depicted in the diagram. Following the cognitive assessment, EEG recordings were conducted during a 2.5-minute baseline resting state with open eyes and a 2.5-minute baseline resting state with closed eyes. Subsequently, either sham or real gTMS stimulation was administered for 40 minutes. During the first 20 minutes of the intervention (“P1”), EEG recordings were conducted, while the F-NAME test was administered at the end of “P2.” After the F-NAME test ended, the stimulation was discontinued and post-gTMS resting state EEG recordings were obtained for 2.5 minutes with open eyes and 2.5 minutes with closed eyes. A post-gTMS cognitive evaluation was performed, following the same protocol as the baseline assessment. In the second session, occurring one week after the first session, a crossover was conducted, where participants switched stimulation arms. Both the baseline and post-cognitive assessments, as well as the intervention and resting state EEG recordings, followed the same design as described for the first session. Times at which EEG recordings were performed are highlighted in purple.

FIG. 5 depicts a graphical representation of the immediate after-effects and short-term electrophysiological changes in the brain induced by gTMS. Participants underwent either 20 minutes of gTMS or a sham stimulation. The gTMS included 110 bursts of 40 Hz TMS with each burst lasting 10 sec. Alterations in gamma, beta, alpha, and theta oscillation power were examined immediately following the stimulation. This analysis involved the exclusion of a specific time window (200 ms before stimulation, 3000 ms duration of stimulation, and 200 ms after stimulation) and the generation and combination of two distinct epochs that were used for subsequent analysis: a baseline epoch (1000-200 ms before stimulation) and the period of interest epoch (200-1000 ms after stimulation). During P1, participants were engaged with a screen throughout the session. Each burst commenced with a countdown from 3 to 1, preparing participants for the upcoming activity. Following the countdown, a “+” sign remained on the screen for the subsequent 6 seconds, providing a consistent visual cue. As the final second approached, the screen transitioned into complete darkness.

FIG. 6 depicts a diagrammatical representation of a flowchart of study participants for gTMS.

FIG. 7A-7B depicts a graphical representation of a spectral analysis conducted following gTMS revealing significant alterations in electrophysiological activity in the brain. FIG. 7A (Left) depicts topographic maps showing localization and direction of significant changes in the average power of theta, alpha, and gamma bands, which occurred immediately after gTMS, as compared to sham stimulation. Electrodes exhibiting a significant increase or decrease in power relative to baseline (dB) are indicated with box points. FIG. 7B (right) depicts time-frequency maps of the electrodes exhibiting a significant difference in average power within both the gamma and theta frequency bands.

FIG. 8 depicts a graphical representation of a spectral analysis conducted during a resting state. The topographic maps illustrate the spatial distribution and direction of changes in the average power of theta, alpha, and gamma bands during resting state EEG analysis for both eyes open and eyes closed conditions.

### DETAILED DESCRIPTION

The present disclosure provides, in part, methods for the treatment, prevention, and/or improvement of neurodegeneration and/or cognitive performance in patients (e.g., older patients) with mild cognitive impairment (MCI), Alzheimer's disease (AD), and/or mild dementia using repetitive transcranial magnetic stimulation (rTMS/gTMS) and/or non-invasive brain stimulation using low intensity magnetic fields at physiologically relevant frequencies.

Alzheimer's disease (AD) is a neurodegenerative disorder characterized by cognitive decline and memory loss. Traditional treatments targeting beta-amyloid accumulation have shown limited success, highlighting the need for alternative therapeutic approaches. Recent studies suggest that disruptions in gamma oscillations may contribute to AD pathology. The disclosed provides, in part, methods of low-intensity gamma magnetic stimulation (referred to herein as both “gTMS” and “rTMS”) on gamma oscillations in AD patients. In embodiments, the intervention is well-tolerated without significant adverse effects. In embodiments, rTMS/gTMS induces significant changes in EEG activity, indicating transient alterations in alpha, beta, theta, and/or gamma frequency band oscillations during and/or immediately after stimulation. In embodiments, low-intensity rTMS/gTMS induces significant changes in gamma oscillations. In embodiments, changes in oscillations (e.g., in gamma oscillations) results in improvement of one or more symptoms of and/or the progression of MCI, AD, MCI to AD conversion, and/or dementia. In embodiments, low-intensity rTMS/gTMS is a safe, non-invasive, long-term treatment approach that subject's and self-apply at home.

Transcranial Magnetic Stimulation (rTMS/gTMS)

In aspects, the present disclosure provides, in part, methods for using non-invasive brain stimulation. In embodiments, the methods include administering repetitive transcranial magnetic stimulation (rTMS/gTMS) by applying one or more magnetic pulses to a surface of the head or a subject in need thereof, where the one or more magnetic pulses is applied at a frequency of less than about 50 Hz and with an intensity of at least about 20,000 milligauss to at least about 250,000 milligauss (e.g., 20-250 gauss, or 2-25 Tesla).

In embodiments, rTMS/gTMS activates and/or inhibits cortical activation by generating a magnetic field that, by Faraday's principle, generates an electrical field inside the brain. In embodiments, the magnetic field is produced when an electric current passes through a coil. Further, in embodiments, to generate an electric field inside the brain, the magnetic field starts and finishes rapidly. Without wishing to be bound by theory, if the magnetic field intensity stays constant too long, then no electrical changes occur inside the brain. In addition, it is contemplated that when making use of magnetic fields of a particular magnitude, neither a voltage transformer nor high currents are necessary. Indeed, the present disclosure contemplates, in part, a device that decreases potential adverse effects, facilitates its transport, and does not require external coolers for the operation, while maintaining a magnetic field that is physiologically useful within the context of MCI, AD, and/or dementia.

rTMS/gTMS, in embodiments, is a non-invasive and non-painful method to stimulate neurons and/or tissue of the brain, including the cerebral cortex, frontal cortex, and/or prefrontal cortex. In embodiments, the present disclosure provides, in part, a device that generates low intensity pulsed magnetic fields at physiologically relevant frequencies. In embodiments, “low intensity,” in reference to a magnetic field/pulse, refers to a strength and/or intensity of a magnetic field in the range of about or at least about 20,000 milligauss to at least about 250,000 milligauss (e.g., 20-250 gauss, or 2-25 Tesla). In embodiments, “physiologically relevant frequency,” in reference to a magnetic field/pulse, refers to a frequency in the range of less than about 50 Hz.

In embodiments, rTMS/gTMS is applied via a magnetic pulse. In embodiments, a “magnetic pulse” refers to the application of a magnetic field for a period of time. In embodiments, one or more magnetic pulses can be applied for methods described herein, e.g., as a single pulse repeated several times with some manner of periodicity, or as several pulses having a first periodicity repeated as a set of pulses having a second periodicity.

In embodiments, rTMS/gTMS includes applying one or more magnetic pulses, where one or more magnetic pulses is applied at a frequency of less than about 50 Hz. In embodiments, the frequency is less than about 0.1 Hz, less than about 0.5 Hz, less than about 1.0 Hz, less than about 2.0 Hz, less than about 3.0 Hz, less than about 4.0 Hz, less than about 5.0 Hz, less than about 6.0 Hz, less than about 7.0 Hz, less than about 8.0 Hz, less than about 9.0 Hz, less than about 10 Hz, less than about 15 Hz, less than about 20 Hz, less than about 25 Hz, less than about 30 Hz, less than about 35 Hz, less than about 40 Hz, less than about 45 Hz, less than about 50 Hz.

In embodiments, the frequency has a range of about 0.1 Hz to about 1.0 Hz, about 1.0 Hz to about 5.0 Hz, about 5.0 Hz to about 10 Hz, about 0.1 Hz to about 10 Hz, about 10 Hz to about 15 Hz, about 15 Hz to about 20 Hz, about 20 Hz to about 25 Hz, about 25 Hz to about 30 Hz, about 20 Hz to about 30 Hz, about 35 Hz to about 40 Hz, about 40 Hz to about 45 Hz, about 30 Hz to about 40 Hz, about 45 Hz to about 50 Hz, or about 40 Hz to about 50 Hz.

In embodiments, the frequency has a range that overlaps with one or more human brain oscillation frequency bands, for example, the frequency has a range of about 4 Hz to about 7 Hz (e.g., theta frequency band), a range of about 8 Hz to about 14 Hz (e.g., alpha frequency band), a range of about 12.5 Hz to about 30 Hz (e.g., beta frequency band), or a range of about 30 Hz to about 50 Hz (e.g., gamma frequency band).

In embodiments, rTMS/gTMS includes applying one or more magnetic pulses at an intensity of about or at least about 20,000 milligauss (20 gauss) to about or at least about 250,000 milligauss (250 gauss). In embodiments, the intensity is about or at least about 20,000 milligauss (20 gauss), about 30,000 milligauss (30 gauss), about 40,000 milligauss (40 gauss), about 50,000 milligauss (50 gauss), about 60,000 milligauss (60 gauss), about 70,000 milligauss (70 gauss), about 80,000 milligauss (80 gauss), about 90,000 milligauss (90 gauss), about 100,000 milligauss (100 gauss), least about 120,000 milligauss (120 gauss), about 130,000 milligauss (130 gauss), about 140,000 milligauss (140 gauss), about 150,000 milligauss (150 gauss), about 160,000 milligauss (160 gauss), about 170,000 milligauss (170 gauss), about 180,000 milligauss (180 gauss), about 190,000 milligauss (190 gauss), about 200,000 milligauss (200 gauss), about 210,000 milligauss (210 gauss), about 220,000 milligauss (220 gauss), about 230,000 milligauss (230 gauss), about 240,000 milligauss (240 gauss), about 250,000 milligauss (250 gauss) or more.

In embodiments, the intensity has a range of about 20,000 to about 25,000 milligauss, about 25,000 to about 30,000 milligauss, about 30,000 to about 35,000 milligauss, about 35,000 to about 40,000 milligauss, about 45,000 to about 50,000 milligauss, about 55,000 to about 60,000 milligauss, about 65,000 to about 70,000 milligauss, about 75,000 to about 80,000 milligauss, about 85,000 to about 90,000 milligauss, about 95,000 to about 100,000 milligauss, about 20,000 to about 100,000 milligauss, about 100,000 to about 120,000 milligauss, about 120,000 to about 140,000 milligauss, about 140,000 to about 160,000 milligauss, about 160,000 to about 180,000 milligauss, about 180,000 to about 200,000 milligauss, about 100,000 to about 200,000 milligauss, or about 200,000 to about 250,000 milligauss or more.

In embodiments, the intensity has a range of about 0.002 Tesla to about 0.025 Tesla or more.

In embodiments, the one or more magnetic pulses generate an electric field of about or at least about 0.1 V/m² to about or at least about 10 V/m², about or at least about 0.5 V/m² to about or at least about 5 V/m², or about or at least about 0.5 V/m² to about or at least about 1.5 V/m². In embodiments, the one or more magnetic pulses generate an electric field of about or at least about 1 V/m², about or at least about 2 V/m², about or at least about 3 V/m², about or at least about 4 V/m², about or at least about 5 V/m², about or at least about 6 V/m², about or at least about 7 V/m², about or at least about 8 V/m², about or at least about 9 V/m², about or at least about 10 V/m², about or at least about 20 V/m², or about or at least about 30 V/m².

In embodiments, the rTMS/gTMS treatment method is performed, applied, or otherwise experienced by a subject at least once daily, at least twice daily, at least thrice daily, or at least four times daily.

In embodiments, the rTMS/gTMS treatment method is performed for greater than about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In embodiments, the method is performed for about 15 to about 60 minutes. In embodiments, the method is performed for about 30 minutes.

In embodiments, the rTMS/gTMS treatment method includes one or more magnetic pulses that are applied about 100 to about 1000 times, or about 200 to about 900 times, or about 200 to about 800 times, or about 200 to about 700 times, or about 200 to about 600 times, or about 200 to about 500 times, or about 200 to about 400 times, or about 200 to about 300 times, or about 300 to about 1000 times, or about 300 to about 900 times, or about 300 to about 800 times, or about 300 to about 700 times, or about 300 to about 600 times, or about 300 to about 500 times, or about 300 to about 400 times.

In embodiments, the rTMS/gTMS method includes one or more magnetic pulses that are applied about 300 to about 400 times. In embodiments, the one or more magnetic pulses are applied about 300 times, about 310 times, about 320 times, about 330 times, about 340 times, about 350 times, about 360 times, about 370 times, about 380 times, about 390 times, or about 400 times.

In embodiments, the rTMS/gTMS method includes one or more magnetic pulses that are applied about 100 to about 150 times. In embodiments, the one or more magnetic pulses are applied about 100 times, about 110 times, about 120 times, about 130 times, about 140 times, or about 150 times. In embodiments, the one or more magnetic pulses are applied about 110 times.

In embodiments, the one or more magnetic pulses are applied discontinuously.

In embodiments, the one or more magnetic pulses last for a period (e.g., a burst) of at least about 2 seconds, at least about 4 seconds, at least about 6 seconds, at least about 8 seconds, at least about 10 seconds, at least about 12 seconds, at least about 14 seconds, at least about 16 seconds, at least about 18 seconds, or at least about 20 seconds. In embodiments, the one or more magnetic pulses last for at least about four seconds. In embodiments, the one or more magnetic pulses last for a period of at least about 10 seconds.

In embodiments, the period of pulsing is followed by a period without pulsing. For example, in embodiments, one or more magnetic pulses are applied discontinuously for a period of about four seconds, followed by a period of about one second without a magnetic pulse. In embodiments, the one or more magnetic pulses last for a period of about 10 seconds followed by a period of at least about 1 second without a magnetic pulse.

In embodiments, the rTMS/gTMS device is configured to provide various patterns of magnetic pulsing and pausing, for example, where periods of discontinuous pulsing (e.g., at less than about 50 Hz) can occur over a period of time (e.g., for about 1 second to about 1 min) with a period of time where the pulsing stops (e.g., for about 1 second to about 1 min).

In embodiments, the rTMS/gTMS is applied chronically. For example, given the nature of MCI, AD, and dementia, in embodiments, the rTMS/gTMS is applied over long periods of time, such as through the life of the subject after diagnosis. In embodiments, the rTMS/gTMS is applied for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, or at least about 10 years or longer. In embodiments, the rTMS/gTMS is applied chronically until one or more symptoms of MCI, AD, and/or dementia, such as cognitive deficits, improve and/or diminish to a degree that equates to that of a healthy adult of similar age and lifestyle profile (e.g., educational background, etc.). In embodiments, the rTMS/gTMS is self-applied, e.g., where use is expected to become routine self-treatment.

In embodiments, the rTMS/gTMS method contemplated by the present disclosure is applied to the patient until symptoms, such as those described herein, improve or diminish. For example, the patient may undergo the treatment and experience a recovery of cognitive ability and hope to terminate the treatment. The patient may re-start the treatment if cognitive decline reemerges. Alternatively, the patient may undergo the treatment and experience a slowing or halting of cognitive loss, but not an improvement. In such a situation, in embodiments, the patient continues treatment chronically to mitigate or prevent further cognitive loss.

rTMS/gTMS Devices

The present disclosure provides, in part, devices and equipment for rTMS/gTMS and/or performing one or more methods described herein.

In embodiments, the devices and/or equipment for rTMS/gTMS includes an electromagnetic field generator and a coil that emits said electromagnetic field. In embodiments, the magnetic field is produced when an electric current passes through a coil. In embodiments, one or more magnetic pulses of TMS are applied repeatedly with a frequency and a determined magnitude over a cortical area to modulate the cerebral cortex activity. In embodiments, such a method is rTMS/gTMS, and such a device would be configured for rTMS/gTMS.

Persons skilled in the art, upon the benefit of this disclosure in its entirety, will appreciate that the methods of the present disclosure are applied via a device capable of administering transcranial magnetic stimulation. In embodiments, the rTMS/gTMS is applied using a device that is suitable for conducting electric current through a coil, thus generating a magnetic field. In embodiments, the device is suitable for self-application, home use, and is optionally portable.

In embodiments, the device can include an interface for controlling one or more variables of rTMS/gTMS, including in non-limiting examples, a touch screen, buttons, scroll wheel, or computer mouse, and can optionally include one or more “digital health” applications and/or “tele-health” solutions. In embodiments, the device is configured to connect via BLUETOOTH or wireless internet (e.g., Wi-Fi) to a smartphone or other computing device, where a subject can, for example, use a smartphone or computer to control and/or perform the methods described herein.

In embodiments, the device generates a lower intensity pulsed magnetic field, e.g., 20-250 gauss, relative to magnets used for magnetic resonance imaging (MRI) which generates approx. 5,000-15,000 gauss. In embodiments, the device generates pulsed magnetic fields at variable frequencies. In embodiments, the device is a transcranial magnetic stimulation system able to generate pulsed magnetic fields in a frequency range from about 0.1 Hz to 50 Hz. In embodiments, the device is a transcranial magnetic stimulation system that generates a magnetic field with an intensity from 20,000 milligauss to about 250,000 milligauss.

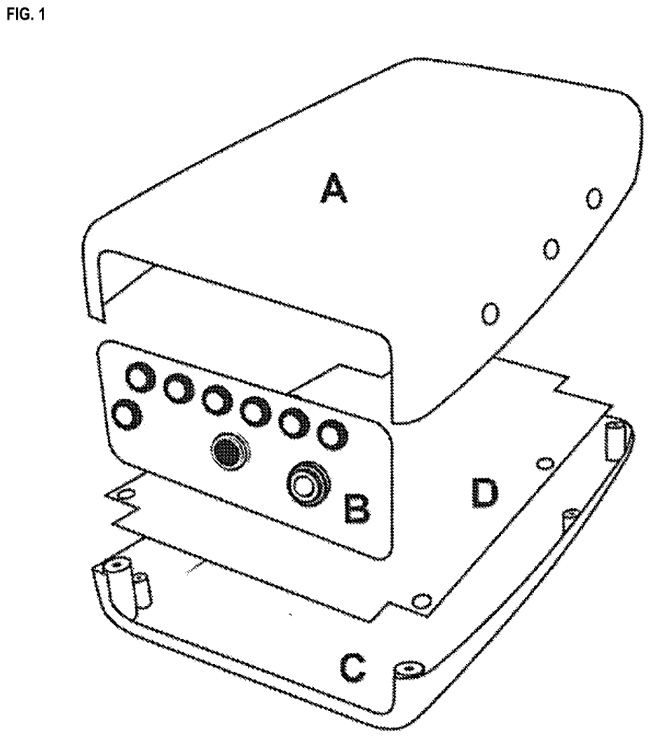

In embodiments, the rTMS/gTMS-generating device includes at least parts. For example, in embodiments and in reference to FIG. 1, the first part of the device comprises an electronic unit that is housed in box (e.g., a plastic box) with another three parts (A), (B), (C), where the electronic board (D) is housed within the center of the box. In embodiments, the box has approx. six sides/faces. In embodiments, a first face, e.g., part (B), is a control panel housing a series of light-emitting diodes (LED), a connector configured to electronically connect with a plug from the coil cord which transmits the electromagnetic waves. In embodiments, one or more faces of the device can have holes for ventilation. In embodiments, at least one face includes a connector configured to electronically connect with a plug of a power adapter cable. In embodiments, one or more faces display information, e.g., a company logo, power requirements, instructions, warnings, etc. In embodiments, the one or more faces are smooth, rough, or have varying surfaces features, e.g., to assist in set up or placement. In embodiments, at least one face, e.g., a back face, has a plastic overhang, clip, or other means to affix the device to a subject's clothes, e.g., to function as a wearable device.

In embodiments, the electronics inside of the device includes a PCB (printed circuit board) and/or CCA (circuit card assembly), in which the electronic components responsible for providing the electromagnetic waves to the coil by the connector are soldered. In embodiments, a light-emitting diode (LED) is turned on to show the device is connected to the power adapter, e.g., such as a green light-emitting diodes (LED). In embodiments, the device is powered by a 12 V, 1 Amp external power supply configured to electrically connect and draw power from a 110V/220V socket. In embodiments, the device includes a series of one or more LEDs, e.g., such as the seven which are horizontally placed in the part (B) (cover) of FIG. 1; these LEDs turn on serially to show that the device is working and/or that a magnetic field is being generated. In embodiments, the one or more LEDs can be configured to visually communicate the progress of the rTMS/gTMS pulsing.

In embodiments, the device includes at least one device haptic which allows for powering the device on and off, e.g., such as an ON/OFF button, trigger, switch, touchscreen, etc., that is in electronic communication with an electronic circuit and can allow the user to start a treatment session, alter the frequency, magnetic strength, pulsing pattern, and/or duration. In embodiments, the electronic circuit is composed of at least three parts: (1) a pulse generator from 0.1 to 50 Hz, (2) an interruption generator, and (3) control of light-emitting diodes (LEDs).

In embodiments, the electronic circuit is composed of four parts: 1) a pulse generator, which generates magnetic pulses from about or at least about 0.1 Hz to about 50 Hz, 2) a current amplifier, 3) a controller for monitoring critical variables, and 4) a battery charging circuit. In embodiments, the electric current passes about or at least about 0.1 times per second to about 50 times per second through one or more coils, where the flux is not substantially interrupted during the duration of the stimulation session. In embodiments, the circuit is able to generate a pulsed magnetic field with an approximate intensity of about or at least about 20,000 milligauss to about or at least about 250,000 milligauss. In embodiments, the board includes one or more transistor, resistor, heat sink, electrolytic capacitor, and/or diode to generate and/or control the magnetic pulse at a given frequency.

In embodiments, the electric current passes about 50 to 200 times per second through one or more coils and the magnetic flux is interrupted about every 4 to 10 seconds for about 1 second. In embodiments, the circuit is able to generate a pulsed magnetic field with an approximate intensity of about 20,000 milligauss to about 250,000 milligauss.

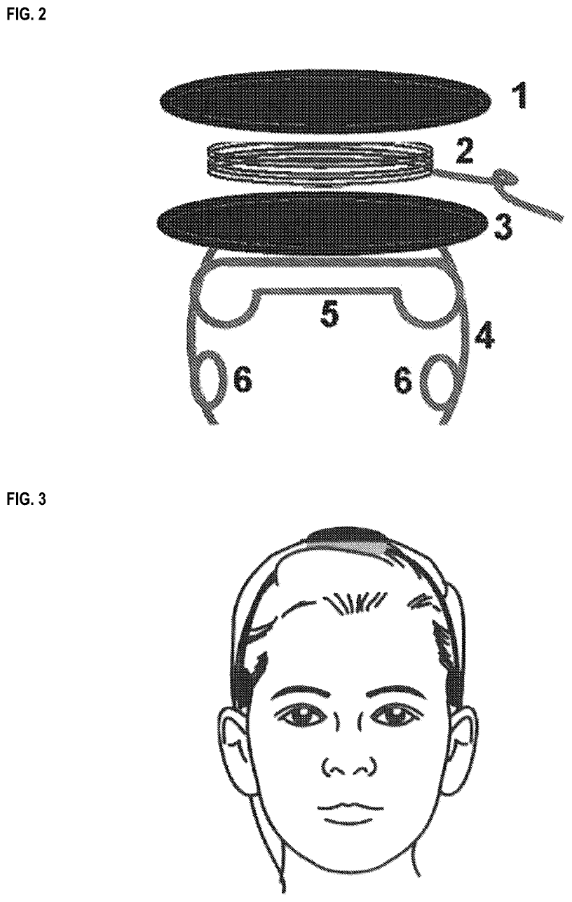

In embodiments, the device includes a second part which is composed of several subparts, e.g., six subparts as depicted in FIG. 2. In embodiments, the device includes an array of coils that transmits the magnetic pulse and/or magnetic field, where the array of coils is located in a helmet configuration that is intended to cover at least a portion of the skull of a subject. In embodiments, the device includes an apparatus to affix the device onto the patient's head, e.g., as shown in FIG. 3. In embodiments, a headband, composed, in part, of steel of approx. 12 cm in diameter (e.g., to fit for any head) is affixed to an inferior portion of the one or more coils. In embodiments, the function of the headband is to transmit magnetic waves throughout the brain volume without the need for a bigger coil. In embodiments, without this element, the electromagnetic waves will reach a short range in the brain. In embodiments, the device comprises a material, e.g., rubber, that fits and/or adapts to the shape of the head of the subject, allowing a soft, comfortable contact with the head. In embodiments, the device includes one or more implements to attach to the head, e.g., such as two movable suction cups to attached to skin of the head.

In embodiments, the device comprises multiple coils of wire having a diameter of about or at least about 1 cm, about or at least about 2 cm, about or at least about 3 cm, about or at least about 4 cm, about or at least about 5 cm, or about or at least about 6 cm. In embodiments, the coils are composed of about or at least about 10 turns of wire, about or at least about 20 turns of wire, about or at least about 30 turns of wire, about or at least about 40 turns of wire, about or at least about 50 turns of wire, about or at least about 100 turns of wire, about or at least about 500 turns of wire, or about or at least about 1000 turns of wire. In embodiments, the number of turns of wire can depend, in part, on the gauge (e.g., thickness) of the wire, the type of material used, and/or the current intended to be passed through the wire. In embodiments, the wire is constructed from a material capable of conducting an electrical current, such as copper. In embodiments, the device comprises one or more coils, where the coil is approx. 5 cm in diameter, and composed of approx. 50 turns of copper wire 99.5% pure, gauge 26 AWG thickness. In embodiments, the coils are insulated, for example a double coverage of insulating material covers the coil winding.

In embodiments, the device is a wearable helmet or headband that includes an array of coils that allows a subject (e.g., the subject intended to receive the rTMS/gTMS, or another person such as a physician) to place the coils in the correct orientation over the skull. In embodiments, the helmet or headband can be fabricated from plastic, steel, carbon fiber, cloth, aluminum, copper, among other materials. In embodiments, the helmet or headband can have one side/surface that is constructed of fabric, silicon, latex, nitrile, and/or rubber e.g., on the side that fits the skull to comfortably fit the subject's head.

In embodiments, the device includes a power source, such as a battery, a set of batteries, or a power cord to draw electrical power from an outlet. In embodiments, the power source is configured to supply the electronics and coil with the necessary electrical current to operate the device and generate the one or more magnetic pulses intended to be applied over the skull and into the brain. In embodiments, the power source, e.g., battery or set of batteries, is configured to be easily changed and/or charged to allow the subject to continue use over longer periods of time (e.g., for a 24-hour period, several times a day or week, etc.).

In embodiments, the device includes a part that comprises a power adapter, e.g., as depicted in FIG. 3. In embodiments, the power adapter provides an alternate current to direct current transformer, and its input is from 120 to 240 Volts with a frequency of 60 Hz, and its output is from 12 V and 2 Amperes.

In embodiments, for example in reference to FIG. 3, the TMS-generating device can include at least three parts: 1) a portable battery pack, 2) an array of coils located inside a helmet, and 3) an electronic unit. In embodiments, the electronic unit comprises a PCB (printed circuit board) in which all the electronic components responsible to generate the electromagnetic waves via the coil remain in electronic communication, e.g., connected via soldering. Those skilled in the art, upon review of this disclosure in its entirety, will appreciate the full spectrum of electronic components that can be used to mechanically and electrically couple a power source to one or more coils for converting electrical power to a magnetic field, as well as how the device can control the magnetic field frequency, periodicity, and amplitude.

Methods of Treatment and/or Prevention of MCI, AD, and/or Dementia

The present disclosure provides, in part, methods of treating, slowing, and/or preventing mild cognitive impairment (MCI), Alzheimer's Disease (AD), and/or dementia, as well as the treating, slowing, and/or preventing the progression of mild cognitive impairment (MCI) to Alzheimer's Disease (AD) and/or to dementia using rTMS/gTMS. In embodiments, the terms “rTMS” and “gTMS” are used interchangeably.

In embodiments, MCI causes cognitive changes that are serious enough to be noticed to the person affected and to family members and friends, but do not affect the individual's ability to carry out everyday activities. Approximately 15% to 20% of people aged 65 and older have varying degrees of MCI. In embodiments, patients presenting with MCI, including MCI involving memory problems, eventually develop AD or other dementias.

In embodiments, the MCI is amnestic MCI, which primarily affects memory. In embodiments, a person having amnestic MCI begins to forget important information that he or she would previously have easily recalled, such as appointments, conversations, or recent events. In embodiments, the MCI is non-amnestic MCI, which affects thinking skills other than memory, including the ability to make sound decisions, judge the time or sequence of steps needed to complete a complex task, or visual perception. In embodiments, risk factors strongly linked to MCI include: advancing age, family history of AD or another dementia, and conditions that raise risk for cardiovascular disease. In embodiments, methods herein for subjects presenting with MCI include practicing forms of prevention against and/or mitigation of MCI, including, but not limited to, controlling blood sugar (e.g., lowering systolic blood pressure), exercise, avoiding smoking and heavy drinking, losing excess weight, limiting caffeine, reducing stress, and eating a diet low in saturated fats and trans fats, cholesterol, and sodium.

In embodiments, the present disclosure provides methods of treating, slowing, and/or preventing Alzheimer's disease (AD). In embodiments, rTMS/gTMS is administered to treat Alzheimer's disease (AD), which is a progressive, neurodegenerative disease characterized in the brain by abnormal clumps (amyloid plaques) and tangled bundles of fibers (neurofibrillary tangles) composed of misplaced proteins. In embodiments, repetitive transcranial magnetic stimulation is used to delay onset, reduce severity, and reduce the rate of progression of AD. In embodiments, rTMS/gTMS is used to treat long-term survivors as well as subjects who exhibit more rapid disease progression. In embodiments, the severity and progression of, and improvement in, AD can be assessed by a number of criteria, including Activities of Daily Living scales, one of which is the Progressive Deterioration Scale (PDS), in which 29 questions are used to determine a subject's ability to perform functions such as interacting socially, traveling without getting lost, coping when the caregiver is away, and identifying or using common household objects such as telephones; the Mini-Mental State Examination (MMSE), which is a short collection of cognitive tests that examines several areas of cognition and is easy to administer and score and is widely used to measure the onset, progression and severity of AD.

In embodiments, age is an important risk factor for AD, where the number of people with the disease doubles every 5 years beyond age 65. Three genes have been discovered that cause early onset (familial) AD. Other genetic mutations that cause excessive accumulation of amyloid protein are associated with age-related (sporadic) AD. In embodiments, methods herein are intended for subjects who are the progeny of a subject with AD, or of a carrier of a mutation in a gene tied to AD, or who are identified with a predisposition of AD (e.g., by a genetic test), are given a prophylactic dose of rTMS/gTMS, e.g., prior to showing physical symptoms of AD.

In embodiment, symptoms of AD include memory loss, language deterioration, impaired ability to mentally manipulate visual information, poor judgment, confusion, restlessness, and mood swings. In embodiments, progression of AD destroys cognition, personality, and the ability to function. In embodiments, subjects suffering from AD experience psychiatric symptoms, including, but not limited to, depression, anxiety, and sleep troubles.

In embodiments, subjects with early AD present with memory deficit, due to AD and/or having AD biomarkers, for example amyloid positive subjects, subjects having a positive florbetapir PET scan, or subjects having a positive Tau PET scan. In embodiments, the early symptoms of AD, which include forgetfulness and loss of concentration, are often overlooked because they resemble natural signs of aging. In embodiments, AD is a progressive disease, but its course can vary from 5 yrs. to 20 yrs., where rTMS/gTMS methods herein are used to ameliorate one or more these symptoms, or to reduce the rate of disease progression, over this time course.

In embodiments, treatments for AD include donepezil (ARICEPT), rivastigmine (EXELON), and galantamine (RAZADYNE), memantine (NAMENDA), and other drugs that may affect AD progression include nonsteroidal anti-inflammatory drugs (NSAIDS), statins, folic acid, Ginkgo biloba, and vitamins E, B6, and B12. In embodiments, one or more of these treatments can be used in combination with rTMS/gTMS.

As the prevalence of AD continues to rise globally (for example, as described in Qiu C, et al. “Epidemiology of Alzheimer's disease: occurrence, determinants, and strategies toward intervention,” Dialogues in Clinical Neuroscience, Vol. 11, No. 2, 2009: pp. 111-28, the entirety of which is hereby incorporated by reference), the search for effective therapeutic interventions becomes increasingly vital. Currently approved pharmacological treatments for AD aim to manage symptoms and target beta-amyloid accumulation in the brain. Nevertheless, even after successfully removing or diminishing these amyloid deposits, the course of neurodegeneration and cognitive decline persists.

In embodiments, cognition in the elderly extends across a spectrum from the normal aging process to dementia. In embodiments, dementia is a severe decline of cognitive function that affects the daily life function of the subject, where this decline exhibits different etiologies, which includes AD, vascular damage, frontotemporal lobar degeneration and Lewy body dementia.

In embodiments, a major risk factor for the development of dementia is age, but dementia is not an inevitable consequence of aging. In embodiments, methods herein are intended for subjects presenting with one or more of the presence of APOE E4 allele, history of traumatic brain injury, depression, heart disease, diabetes mellitus, hypertension, obesity, and tobacco use. In embodiments, methods herein are compatible with treatments that improve neurodegenerative disorders related to dementia. In embodiments, existing treatments for dementia are directed to improve neurotransmission through inhibitors of acetylcholinesterase (donepezil, rivastigmine, galantamine) and inhibitors of NMDA receptors, such as memantine. Although these molecules can have beneficial effects in the stabilization of some cognitive symptoms, they do not modify the prognosis of the disease.

Non-invasive brain stimulation techniques such as transcranial alternating current stimulation (TACS) and repetitive transcranial magnetic stimulation (rTMS/gTMS) have been studied in AD populations showing positive cognitive (for example, as described in Traikapi A, et al. “Episodic memory effects of gamma frequency precuneus transcranial magnetic stimulation in Alzheimer's disease: A randomized multiple baseline study,” J Neuropsychol, Vol. 17, No. 2, 2023: pp. 279-301, the entirety of which is hereby incorporated by reference), and hippocampal perfusion changes (for example, as described in Sprugnoli G, et al. “Impact of multisession 40 Hz tACS on hippocampal perfusion in patients with Alzheimer's disease,” Alzheimers Res Ther, Vol. 13, No. 1, 2021:203, the entirety of which is hereby incorporated by reference). TMS uses electromagnetic induction to generate an electric field inside the brain; classically, TMS devices operate near 1 tesla magnetic fields (1.0×10⁷ milligauss) to induce electric fields of around 100 v/m² in the brain. This approach presupposes that high-intensity fields are necessary for modulating neural activity and to have a measurable effect on neural oscillations.

In embodiments, methods herein utilize a different approach in the treatment of MCI, AD, dementia, and progression therebetween, using physiologically relevant frequency gamma magnetic stimulation to induce changes in gamma oscillations within the brain.

In embodiments, the gTMS method is for treating, slowing, and/or preventing MCI by repetitively applying a magnetic pulse to the scalp of a subject in need thereof, thereby stimulating neurons in the brain of the subject, where the magnetic pulse is applied, a) repetitively over the subject's brain, and b) at a frequency of less than about 50 Hz and an intensity of about 20,000 to about 250,000 milligauss.

In embodiments, the rTMS/gTMS method is for treating, slowing, and/or preventing AD by repetitively applying a magnetic pulse to the scalp of a subject in need thereof thereby stimulating neurons in the brain of the subject, where the magnetic pulse is applied a) repetitively over the subject's brain, and b) at a frequency of less than about 50 Hz and an intensity of about 20,000 to about 250,000 milligauss.

In embodiments, the rTMS/gTMS method is for treating, slowing, and/or preventing a conversion of MCI to AD by repetitively applying a magnetic pulse to the scalp of a subject in need thereof thereby stimulating neurons in the brain of the subject, where the magnetic pulse is applied a) repetitively over the subject's brain, and b) at a frequency of less than about 50 Hz and an intensity of about 20,000 to about 250,000 milligauss.

In embodiments, the rTMS/gTMS method is for treating, slowing, and/or preventing dementia by repetitively applying a magnetic pulse to the scalp of a subject in need thereof thereby stimulating neurons in the brain of the subject, where the magnetic pulse is applied a) repetitively over the subject's brain, and b) at a frequency of less than about 50 Hz and an intensity of about 20,000 to about 250,000 milligauss.

In embodiments, methods herein include administering gTMS by applying one or more magnetic pulses, where one or more magnetic pulses is applied at a frequency of less than about 50 Hz. In embodiments, the frequency is about 0.1 Hz, about 0.5 Hz, about 1.0 Hz, about 2.0 Hz, about 3.0 Hz, about 4.0 Hz, about 5.0 Hz, about 6.0 Hz, about 7.0 Hz, about 8.0 Hz, about 9.0 Hz, about 10 Hz, about 15 Hz, about 20 Hz, about 25 Hz, about 30 Hz, about 35 Hz, about 40 Hz, about 45 Hz, or less than about 50 Hz. In embodiments, the frequency has a range of about 0.1 Hz to about 1.0 Hz, about 1.0 Hz to about 5.0 Hz, about 5.0 Hz to about 10 Hz, about 0.1 Hz to about 10 Hz, about 10 Hz to about 15 Hz, about 15 Hz to about 20 Hz, about 20 Hz to about 25 Hz, about 25 Hz to about 30 Hz, about 20 Hz to about 30 Hz, about 35 Hz to about 40 Hz, about 40 Hz to about 45 Hz, about 30 Hz to about 40 Hz, about 45 Hz to about 50 Hz, or about 40 Hz to about 50 Hz.

In embodiments, methods herein include administering gTMS by applying one or more magnetic pulses at an intensity of about or at least about 20,000 milligauss (20 gauss) to about or at least about 250,000 milligauss (250 gauss). In embodiments, the intensity is about or at least about 20,000 milligauss (20 gauss), about 30,000 milligauss (30 gauss), about 40,000 milligauss (40 gauss), about 50,000 milligauss (50 gauss), about 60,000 milligauss (60 gauss), about 70,000 milligauss (70 gauss), about 80,000 milligauss (80 gauss), about 90,000 milligauss (90 gauss), about 100,000 milligauss (100 gauss), least about 120,000 milligauss (120 gauss), about 130,000 milligauss (130 gauss), about 140,000 milligauss (140 gauss), about 150,000 milligauss (150 gauss), about 160,000 milligauss (160 gauss), about 170,000 milligauss (170 gauss), about 180,000 milligauss (180 gauss), about 190,000 milligauss (190 gauss), about 200,000 milligauss (200 gauss), about 210,000 milligauss (210 gauss), about 220,000 milligauss (220 gauss), about 230,000 milligauss (230 gauss), about 240,000 milligauss (240 gauss), about 250,000 milligauss (250 gauss) or more.

In embodiments, the intensity has a range of about 20,000 to about 25,000 milligauss, about 25,000 to about 30,000 milligauss, about 30,000 to about 35,000 milligauss, about 35,000 to about 40,000 milligauss, about 45,000 to about 50,000 milligauss, about 55,000 to about 60,000 milligauss, about 65,000 to about 70,000 milligauss, about 75,000 to about 80,000 milligauss, about 85,000 to about 90,000 milligauss, about 95,000 to about 100,000 milligauss, about 20,000 to about 100,000 milligauss, about 100,000 to about 120,000 milligauss, about 120,000 to about 140,000 milligauss, about 140,000 to about 160,000 milligauss, about 160,000 to about 180,000 milligauss, about 180,000 to about 200,000 milligauss, about 100,000 to about 200,000 milligauss, or about 200,000 to about 250,000 milligauss or more.

In embodiments, methods herein include administering gTMS by applying a magnetic intensity of a range of about 0.002 Tesla to about 0.025 Tesla or more.

In embodiments, methods herein include administering one or more magnetic pulses to generate an electric field of about or at least about 0.1 V/m² to about or at least about 10 V/m², about or at least about 0.5 V/m² to about or at least about 5 V/m², or about or at least about 0.5 V/m² to about or at least about 1.5 V/m². In embodiments, the one or more magnetic pulses generate an electric field of about or at least about 1 V/m², about or at least about 2 V/m², about or at least about 3 V/m², about or at least about 4 V/m², about or at least about 5 V/m², about or at least about 6 V/m², about or at least about 7 V/m², about or at least about 8 V/m², about or at least about 9 V/m², about or at least about 10 V/m², about or at least about 20 V/m², or about or at least about 30 V/m².

In embodiments, methods of administering gTMS herein modulate one or more of gamma, theta, alpha, and beta activity in the brain. In embodiments, methods of administering gTMS herein modulate gamma activity in the brain. In embodiments, these are specific frequency bands that occur within the brain that are measurable, e.g., by electroencephalography (EEG) recordings. In embodiments, theta is ˜4-7 Hz, alpha is ˜8-14 Hz, beta is ˜12.5-30 Hz, and gamma is ˜30-80 Hz.

In embodiments, methods herein stimulate or otherwise modulate the rhythmic electrical activity, e.g., oscillations, which are a component of brain function. In embodiments, modulations in the oscillations in the gamma-frequency band (˜30-80 Hz) bind neurons into a common temporal matrix, enabling precise temporal coding, spike timing-dependent plasticity, and formation of neuronal assemblies. In embodiments, the gamma oscillations are dependent on precise timing of action potential generation and the finely balanced interplay between excitatory and inhibitory neurotransmission in the neuronal network. In embodiments, the modulation of gamma oscillations effect the higher cognitive functions, such as sensory perception, attention, and memory, which are known to be significantly degraded in AD patients who suffer from deficiencies in their cognitive faculties, for example as described in Buzsáki G & Wang X-J, “Mechanisms of Gamma Oscillations,” Annual Review of Neuroscience, Vol. 35, 2012: pp. 203-25; and Buzsáki G & Watson B O, “Brain rhythms and neural syntax: implications for efficient coding of cognitive content and neuropsychiatric disease,” Dialogues in Clinical Neuroscience, Vol. 14, No. 4, 2012: pp. 345-67, each of which is hereby incorporated by reference in their entirety.

Recent findings suggest that disruption of neural oscillations, specifically in the gamma range, are an important pathological mechanism frequently observed in AD subjects, for example as described in Benussi A, et al. “Increasing Brain Gamma Activity Improves Episodic Memory and Restores Cholinergic Dysfunction in Alzheimer's Disease,” Ann Neurol, Vol. 92, No. 2, 2022: pp. 322-34; Liu C, et al. “Modulating Gamma Oscillations Promotes Brain Connectivity to Improve Cognitive Impairment,” Cerebral Cortex, Vol. 32, 2022: pp. 2644-56; and He Q, et al. “A feasibility trial of gamma sensory flicker for patients with prodromal Alzheimer's disease,” Alzheimers Dement (NY), Vol. 7, No. 1, 2021: e12178, the entirety of each of which is hereby incorporated by reference. Animal model studies have shown that restoring gamma oscillations via sensory stimuli (Buzsáki G & Watson B O, 2012), or optogenetic stimulation (for example, as described in Etter G, et al. “Optogenetic gamma stimulation rescues memory impairments in an Alzheimer's disease mouse model,” Nat Commun, Vol. 10, No. 5322, 2019, the entirety of which is hereby incorporated by reference), improves memory in 5XFAD mice. Moreover, recent pilot human studies using non-invasive entrainment of gamma frequency oscillations using light and sound flickering at 40 Hz have shown promising physiological changes in network connectivity and EEG entrainment at gamma frequencies, for example as describe din He et al., 2021; and Chan D, et al. “Gamma frequency sensory stimulation in mild probable Alzheimer's dementia patients: Results of feasibility and pilot studies,” PLOS One, Vol. 17, No. 12, 2022: e0278412, the entirety of which is hereby incorporated by reference.

Methods herein, in embodiments, use the immediate effects of rTMS/gTMS on brain activity in which one or more sessions of stimulation increase gamma and/or theta oscillation power, where chronic use at lower intensities is beneficial for cognition, memory, etc. In embodiments, physiologically relevant frequency rTMS/gTMS is suitable for longer interventions for modulation of gamma oscillations in subjects with AD and/or for sustained gamma modulation and improved cognition, for instance as described in Liu et al. “Modulating Gamma Oscillations Promotes Brain Connectivity to Improve Cognitive Impairment,” Cerebral Cortex, Vol. 32, 2022: pp. 2644-56, hereby incorporated by reference in its entirety, which demonstrated the strengthening of gamma-band power at the left temporoparietal cortex and improved cognitive function eight weeks after a 12-session gamma rTMS/gTMS stimulation protocol. In embodiments, methods herein induce electrophysiological changes that affect episodic memory, e.g., following 10-day or longer rTMS/gTMS intervention sessions targeting the precuneus. In embodiments, methods herein include applying rTMS/gTMS for modulation of gamma frequencies after multiple rTMS/gTMS sessions to observe positive changes in cognition under, e.g., low intensity and physiologically relevant frequencies.

Methods herein, in embodiments, modulate theta-gamma coupling, a process implicated in various cognitive functions such as memory formation, attention, and information processing. Notably, disruptions in theta-gamma coupling have been observed in animal models and individuals with AD, underlining the potential relevance of gamma neuromodulation in AD physiopathology, for example as described in Goutagny R, et al., “Alterations in hippocampal network oscillations and theta-gamma coupling arise before Aβ overproduction in a mouse model of Alzheimer's disease,” European Journal of Neuroscience, Vol. 37, 2013: pp. 1896-902; and Goodman M S, et al., “Theta-Gamma Coupling and Working Memory in Alzheimer's Dementia and Mild Cognitive Impairment,” Frontiers in Aging Neuroscience, Vol. 10, No. 101, 2018, each of which is hereby incorporated by reference in their entirety.

In embodiments, methods of administering rTMS/gTMS improve the ability of therapeutics to cross the blood-brain barrier (BBB), for example, for treatments targeted to dissolve or solubilize proteinaceous aggregates, for example as described in Kurudenkandy F R, et al. “Amyloid-β-Induced Action Potential Desynchronization and Degradation of Hippocampal Gamma Oscillations Is Prevented by Interference with Peptide Conformation Change and Aggregation,” J Neurosci, Vol. 34, 2014: pp. 11416-25, the entirety of which is hereby incorporated by reference.

In embodiments, methods of rTMS/gTMS are performed, applied, or otherwise experienced by a subject at least once daily, at least twice daily, at least thrice daily, or at least four times daily.

In embodiments, the rTMS/gTMS treatment method is administered, e.g., in one or more sessions, lasting for greater than about 15 min. In embodiments, the rTMS/gTMS methods are administered for about 15 minutes, about 20 minutes, about 25 minutes, about 30 minutes, about 35 minutes, about 40 minutes, about 45 minutes, about 50 minutes, about 55 minutes, or about 60 minutes or more. In embodiments, the rTMS/gTMS methods are administered for about 15 to about 60 minutes. In embodiments, the rTMS/gTMS methods are administered for about 30 minutes.

In embodiments, rTMS/gTMS methods herein include applying one or more magnetic pulses that are applied about 100 to about 1000 times, or about 200 to about 900 times, or about 200 to about 800 times, or about 200 to about 700 times, or about 200 to about 600 times, or about 200 to about 500 times, or about 200 to about 400 times, or about 200 to about 300 times, or about 300 to about 1000 times, or about 300 to about 900 times, or about 300 to about 800 times, or about 300 to about 700 times, or about 300 to about 600 times, or about 300 to about 500 times, or about 300 to about 400 times. In embodiments, the TMS/gTMS method includes one or more magnetic pulses that are applied about 300 to about 400 times. In embodiments, the one or more magnetic pulses are applied about 300 times, about 310 times, about 320 times, about 330 times, about 340 times, about 350 times, about 360 times, about 370 times, about 380 times, about 390 times, or about 400 times.

In embodiments, the rTMS/gTMS method includes one or more magnetic pulses that are applied about 100 to about 150 times. In embodiments, the one or more magnetic pulses are applied about 100 times, about 110 times, about 120 times, about 130 times, about 140 times, or about 150 times. In embodiments, the one or more magnetic pulses are applied about 110 times.

In embodiments, the one or more magnetic pulses are applied discontinuously.

In embodiments, the one or more magnetic pulses last for a period (e.g., a burst) of at least about 2 seconds, at least about 4 seconds, at least about 6 seconds, at least about 8 seconds, at least about 10 seconds, at least about 12 seconds, at least about 14 seconds, at least about 16 seconds, at least about 18 seconds, or at least about 20 seconds. In embodiments, the one or more magnetic pulses last for at least about four seconds. In embodiments, the one or more magnetic pulses last for a period of at least about 10 seconds.

In embodiments, the period of pulsing is followed by a period without pulsing. For example, in embodiments, one or more magnetic pulses are applied discontinuously for a period of about four seconds, followed by a period of about one second without a magnetic pulse. In embodiments, the one or more magnetic pulses last for a period of about 10 seconds followed by a period of at least about 1 second without a magnetic pulse.

In embodiments, the rTMS/gTMS device is configured to provide various patterns of magnetic pulsing and pausing, for example, where periods of discontinuous pulsing (e.g., at less than about 50 Hz) can occur over a period of time (e.g., for about 1 second to about 1 min) with a period of time where the pulsing stops (e.g., for about 1 second to about 1 min).

In embodiments, rTMS/gTMS methods herein include are applied chronically and/or long-term. For example, given the nature of MCI, AD, and dementia, in embodiments, the rTMS/gTMS methods are applied over long periods of time, such as throughout the life of the subject after a diagnosis. In embodiments, the rTMS/gTMS method is applied for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, or at least about 10 years or longer. In embodiments, the rTMS/gTMS methods are applied chronically until one or more symptoms of MCI, AD, and/or dementia, such as cognitive deficits, improve and/or diminish to a degree that equates to that of a healthy adult of similar age and lifestyle profile (e.g., educational background, etc.). In embodiments, the rTMS/gTMS is self-applied, e.g., where use is expected to become routine self-treatment.

In embodiments, the rTMS/gTMS methods are intended to be administered for long-term or chronic use for cognitive and/or behavioral improvement. In embodiments, the rTMS/gTMS methods are administered at least once daily, twice daily, thrice daily, or at least four times daily. In embodiments, the gTMS methods are performed twice daily. In embodiments, rTMS/gTMS methods are administered daily, once every two days, once every three days, once every four days, once every five days, once every six days, once per week, once biweekly, once every three weeks, or once per month. In embodiments, the rTMS/gTMS methods are administered with or timed alongside a therapeutic agent (e.g., synchronously or asynchronously), for example administered prior to administration of a therapeutic agent, after administration of a therapeutic agent, and/or substantially simultaneously with administration of a therapeutic agent.

In embodiments, methods herein are suitable for self-application. In embodiments, the one or more magnetic pulses are applied using a device configured for conducting an electric current through one or more coils, e.g., as described herein. In embodiments, methods herein are suitable for home use. In embodiments, methods herein use a rTMS/gTMS device that is portable and/or wearable, for example, as depicted in FIGS. 2-3, where the device functions as a headband, helmet, or other wearable device.

Methods herein, in embodiments, are administered to a subject that is afflicted with, diagnosed with, or suspected of a diagnosis of mild cognitive impairment (MCI). Methods herein, in embodiments, are administered to a subject that is afflicted with, diagnosed with, or suspected of a diagnosis of amnestic type MCI. Methods herein, in embodiments, are administered to a subject that is afflicted with, diagnosed with, or suspected of a diagnosis of non-amnestic type MCI.

Methods herein, in embodiments, are administered to a subject that is afflicted with, diagnosed with, or suspected of a diagnosis of preclinical Alzheimer's disease (AD). Methods herein, in embodiments, are administered to a subject that is afflicted with, diagnosed with, or suspected of a diagnosis of mild AD. Methods herein, in embodiments, are administered to a subject that is afflicted with, diagnosed with, or suspected of a diagnosis of moderate AD.

Methods herein, in embodiments, are administered to a subject that is afflicted with, diagnosed with, or suspected of a diagnosis of dementia (e.g., mild dementia).

In embodiments, methods herein include measuring and/or assessing one or more biomarkers indicative of AD. In embodiments, the biomarker includes amyloid beta (Aβ) in cerebrospinal fluid (e.g., detecting high Aβ in cerebrospinal fluid), Tau protein in cerebrospinal fluid (e.g., detecting high total Tau (t-tau) protein levels and/or phosphorylated Tau (p-Tau) in cerebrospinal fluid), and detecting the presence of the ApoE4 allele (e.g., including any single nucleotide polymorphisms (SNPs), mutations, aberrations in expression, etc.). Methods herein, in embodiments, are administered to a subject that presents as having at least one biomarker indicative of AD, e.g., including high AB in cerebrospinal fluid, high Tau in cerebrospinal fluid, and the presence of the ApoE4 allele.

In embodiments, methods herein include measuring and/or assessing one or more biomarkers of MCI, AD, and/or dementia prior to and/or after one or more sessions of rTMS/gTMS.

In embodiments, measuring and/or assessing one or more biomarkers includes immunohistochemical staining, western blotting, in cell western, immunofluorescent staining, ELISA, fluorescent activating cell sorting (FACS), DNA sequencing, RNA sequencing, polymerase chain reaction (PCR), reverse transcriptase polymerase chain reaction (RT-PCR), quantitative PCR (qPCR), multiplex polymerase chain reaction, nested polymerase chain reaction, hot start polymerase chain reaction, long-range PCR, assembly polymerase chain reaction, asymmetric polymerase chain reaction, or digital droplet PCR, RNA or DNA gel electrophoresis or Southern or Northern blotting, and/or a microarray-based assay, or a combination thereof. In embodiments, the evaluating is performed by contacting a sample of cerebrospinal fluid with an agent that specifically binds to or more of the nucleic acids encoding the biomarker and/or a nucleic acid complementary thereto, and/or that binds the biomarker protein.

In embodiments, methods herein include measuring and/or assessing one or more symptoms of the AD and/or MCI, wherein the measuring and/or assessing occurs prior to applying the rTMS/gTMS and/or after applying the rTMS/gTMS. In embodiments, methods herein include evaluating or otherwise measuring and/or assessing cognitive and/or behavioral improvement. In embodiments, the measuring and/or assessing includes one or more tests, scales, and/or methods, e.g., as listed in Table 1.

TABLE 1
Exemplary tests, scales, and/or methods used for the assessment of mild cognitive impairment
(MIC), Alzheimer's Disease (AD), conversions of MCI to AD, and/or dementia pathology.

| Aspects of AD Disorder | Abbreviation | Test/Scale Name |
|---|---|---|
| Cognitive Impairment | ADAS-Cog | Alzheimer's Disease Assessment Scale - Cognitive |
| | | Subscale |
| | BIMC | Blessed Information-Memory-Concentration Test |
| | CAMCOG | Cambridge Cognitive Examination |
| | F-NAME | Face-Name Associative Memory Exam test |
| | MDRS | Mattis Dementia Rating Scale |
| | MMSE | Mini-Mental State Examination |
| | NTB | Neuropsychological Test Battery |
| | pVFT | Phonetic Verbal Fluency Test |
| | RAVLT | Rey Auditory Verbal Learning Test |
| | sVFT | Semantic Verbal Fluency Test |
| | SCIP | Severe Impairment Profile |
| | SIB | Severe Impairment Profile |
| | SKT | Syndrom-Kuztest |
| Activities of Daily Living | ADL-P | Activities of Daily Living-Prevention Instrument |
| | ADLQ | Activities of Daily Living Questionnaire |
| | ADCS-ADL19/23 | Alzheimer's Disease Cooperative Study Activities of |
| | | Daily Living Inventory 19- or 23-item scale |
| | B-ADI | Bayer Activities of Daily Living Scale |
| | Bristol ADL | Bristol Activities of Daily Living Scale |
| | BANS-S | Bedfords Alzheimer's Nursing Severity Scale |
| | Barthel ADL | Barthel Activities of Daily Living Index Scale |
| | DAD | Disabilities Assessment for Dementia |
| | GERRI | Geriatric Evaluation by Relative's Rating Instrument |
| | IDDD | Interview for Deterioration in Daily Living Activities in |
| | | Dementia |
| | Katz ADL | The Katz Index of Independence in Activities of Daily |
| | | Living |
| | PGDRS | Psychogeriatric Dependency Rating Scales |
| | RDRS | Rapid Disability Rating Scale |
| | PSMS | Physical Self-Maintenance Scale |
| | SMAF | Functional Autonomy Measurement System |
| | Weintraub ADL | Weintraub Activities of Daily Living Scale |
| Global Impression | ADCS-CGIC | Alzheimers' Disease Cooperative Study - Clinical |
| | | Global Impression of Change |
| | CDR | Clinical Dementia Rating Scale |
| | CGI | Clinical Global Impression |
| | CIBI | Clinical Interview-Based Impression (Parke-Davis) |
| | FAST | The Functional Assessment Staging |
| | GDS | Global Deterioration Scale |
| | HDR | Hierarchic Dementia Scale |
| | NYU CIBIC-Plus | The New York University - Clinician's Interview-Based |
| | | Impression of Change - Plus Caregiver Input |
| Behavior-Neuropsychology | AMS | Alzheimer's Mood Scale |
| | IA | Apathy Inventory |
| | BEAM-D | Behavioral and Emotional Activities Manifested in |
| | | Dementia |
| | BEHAVE-AD | Behavioral Pathology in Alzheimer's Disease Rating |
| | | Scale |
| | BPRS | Brief Psychiatric Rating Scale |
| | CERAD-BRSD | Behavior Rating Scale for Dementia of the Consortium |
| | | to Establish a Registry for Alzheimer's Disease |
| | CMAI | Cohen-Mansfield Agitation Inventory |
| | CSDD | Cornell Scale for Depression in Dementia |
| | DBD | Dementia Behavior Disturbance Scale |
| | DMAS | Dementia Mood Assessment Scale |
| | GDS | Geriatric Depression Scale |
| | HoNOS 65+ | Health of the Nation Outcome Scale for Elderly People |
| | NPI | Neuropsychiatric Inventory |
| | NRS | Neurobehavioral Rating Scale |
| | RMBPC | Revised Memory and Behavior Problems Checklist |
| Quality of Life | ADRQL | Alzheimer's Disease Related Quality of Life |
| | DQoL | Dementia Quality of Life Instrument |
| | DS-DAT | Discomfort Scale for Dementia of Alzheimer's Type |
| | OERS | Observed Emotion Rating Scale |
| | PDS | Progressive Deterioration Scale |
| | QoL-AD | Quality of Life-Alzheimer's Disease |
| | QUALID | Quality of Life in Late-Stage Dementia Scale |
| Communication/Social | CLB | Core Linguistic Battery |
| Interaction | CPS | Communication Problems Scale |
| | ERT | Emotion Recognition Scale |
| | MOSES | Multidimensional Observation Scale for Elderly |
| | | Subjects |
| | PKT | Posture Knowledge Test |
| | PPQSA | Partner-Patient Questionnaire for Shared Activities |
| | SCS | Social Competence Scale |
| Multi-Domain | BGP | Behavioral Rating Scale for Geriatric Patients |
| | GBS | Gottfries-Brane-Steen Scale |
| | NOSGER | Nurses' Observation Scale for Geriatric Patients |
| | SCAG | Sandoz Clinical Assessment Geriatric Scale |
| | VL | The Vienna List |

In embodiments, the measuring and/or assessing is performed using a Face-Name Associative Memory Exam (F-NAME) test, Phonetic Verbal Fluency Test (pVFT), Semantic Verbal Fluency Test (SVFT), Rey Auditory Verbal Learning Test (RAVLT), Montreal Cognitive Assessment (MoCA), Alzheimer's Disease Assessment Scale-Cognitive Subscale (ADAS-Cog), Clinical Dementia Rating (CDR), and/or a Clinical Dementia Rating Scale-Sum of Boxes (CDR-SB).

In embodiments, the subject presents as having a test score of one or more identified instruments for assessment of AD and/or MCI symptoms that is considered to indicate the presence of cognitive impairment or AD.

In embodiments, the one or more identified instruments for assessment of AD and/or MCI symptoms is a Face-Name Associative Memory Exam (F-NAME). In embodiments, the subject present as having a F-NAME test score of less than about 25 (e.g., total score: Face+Letter+Name).

In embodiments, the one or more identified instruments for assessment of AD and/or MCI symptoms is a Montreal Cognitive Assessment (MoCA). In embodiments, the MoCA assesses: short term memory; visuospatial abilities; executive functions; attention; concentration and working memory, language; and orientation to time and place. In embodiments, MoCA includes a scale that ranges from 0-30 (e.g., in increments of 0.01), where scores of 26+ are considered normal. In embodiments, the subject present as having a MoCA test score of less than about 26. In an illustrative study, people without cognitive impairment scored an average of 27.4; people with MCI scored an average of 22.1; people with Alzheimer's disease scored an average of 16.2. The short-term memory recall task (5 points) involves two learning trials of five nouns and delayed recall after approximately five minutes. Visuospatial abilities are assessed using a clock-drawing task (3 points) and a three-dimensional cube copy (1 point). Multiple aspects of executive functions are assessed using an alternation task adapted from the trail-making B task (1 point), a phonemic fluency task (1 point), and a two-item verbal abstraction task (2 points). Attention, concentration, and working memory are evaluated using a sustained attention task (target detection using tapping; 1 point), a serial subtraction task (3 points), and digits forward and backward (1 point each). Language is assessed using a three-item confrontation naming task with low-familiarity animals (3 points), repetition of two syntactically complex sentences (2 points), and the fluency task. Abstract reasoning is assessed using a “describe the similarity” task with 2 points being available. Orientation to time and place is evaluated by asking the subject for the date and the city in which the test is occurring (6 points).

In embodiments, treatment with the rTMS/gTMS methods described herein result in an improvement (i.e., an increase) in the patient's MoCA score compared to a baseline (e.g., prior to treatment, or at an earlier timepoint during treatment). In embodiments, the subject's MoCA score increases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of decrease in their MoCA score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable MoCA score compared to a baseline (e.g., prior to treatment or at an earlier timepoint during treatment), which is indicative of a slowing, delay, or cessation of the progression of AD, or a lack of appearance of new clinical, functional, or cognitive symptoms or impairments, or an overall stabilization of disease activity.

In embodiments, the one or more identified instruments for assessment of AD and/or MCI symptoms is an Alzheimer's Disease Assessment Scale-Cognitive Subscale (ADAS-Cog). In embodiments, the ADAS-Cog is used to assess the severity of cognitive symptoms of dementia. The ADAS-Cog is considered a “gold standard” for assessing the efficacy of antidementia treatments. In embodiments, the full ADAS takes about 45 minutes to administer, and is scored from 0 to 150 by summing the number of errors made on each task so that higher scores indicate worse performance. In embodiments, this instrument allows exploring the cognitive processes commonly affected in Alzheimer's disease, including 11 items that can be scored from 0 to 5, having a maximum of 70 points, the higher the score, the greater the degree of alteration. The concurrent validity with the Dementia Rating Scale was r=0.642 (p<0.001), the interobserver reliability was 0.650-0.989 (p<0.01) for patients with Alzheimer's disease and 0.658-1 (p<0.01) for cognitively normal, the test-retest reliability was 0.579 (p<0.05)-0.919 (p<0.001). Considering an adjustment of the age and education level score, the cut-off point of the ADAS-Cog was ≥12 (sensitivity: 89-19%, specificity: 88.53%), the area under the ROC curve was 0.95, 0.94. In embodiments, the version of the ADAS-Cog includes 13 items (which is referred to as “ADAS-Cog 13”), for example as described in Rosen et al., 1984, Amer. J. Psych. Vol. 141: pp. 1356-1364; Mohs et al., 1997, Alzheimer's disease Assoc. Disorders, 11 (2): S13-S21, each of which is hereby incorporated by reference in their entirety. In embodiments, the subject presents as having a ADAS-Cog test score of 2, 3, 4, or 5 on one or more subscores of the test.

In embodiments, methods herein include measuring and/or assessing using the ADAS-Cog 13, which is a multi-part cognitive assessment that assesses multiple cognitive domains, including memory, naming, word finding, comprehension, praxis, attention, orientation, and spontaneous speech. In embodiments, the ADAS-Cog 13 is based on the ADAS-Cog and additionally includes delayed word recall and a number cancellation task. In embodiments, the ADAS-Cog 13 produces scores up to 85 points. In embodiments, the subject for methods herein presents as having a ADAS-Cog test score of 2, 3, 4, or 5 on one or more subscores of the test. In embodiments, a decrease in ADAS-Cog 13 score is indicative of improvement in the subject's condition, whereas an increase in ADAS-Cog 13 score is indicative of worsening of the subject's condition. In embodiments, a stable ADAS-Cog 13 score is indicative of a slowing, delay, or cessation of the progression of MCI, AD, dementia, and/or a reduction in clinical or cognitive decline.

In embodiments, treatment with the rTMS/gTMS methods described herein result in an improvement (i.e., a decrease) in the subject's ADAS-Cog score (e.g., ADAS-Cog 13) compared to a baseline (e.g., prior to treatment or at an earlier timepoint during treatment). In embodiments, the subject's ADAS-Cog score decreases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of increase in their ADAS-Cog score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable ADAS-Cog score compared to a baseline (e.g., prior to treatment or at an earlier timepoint during treatment), which is indicative of a slowing, delay, or cessation of the progression of AD, or a reduction in clinical or cognitive decline.

In embodiments, the one or more identified instruments for assessment of AD, MCI, and/or dementia symptoms is a Clinical Dementia Rating (CDR) and/or a Clinical Dementia Rating Scale-Sum of Boxes (CDR-SB). In embodiments, the Clinical dementia rating (CDR) is calculated on the basis of testing six different cognitive and behavioral domains such as memory, orientation, judgment and problem solving, community affairs, home and hobbies performance, and personal care. In embodiments, the CDR is based on a scale of 0-3: no dementia (CDR=0), questionable dementia (CDR=0.5), MCI (CDR=1), moderate cognitive impairment (CDR=2), and severe cognitive impairment (CDR=3).

In embodiments, treatment with the rTMS/gTMS methods described herein result in an improvement (i.e., a decrease) in the subject's CDR score compared to a baseline (e.g., prior to treatment or at an earlier timepoint during treatment). In some embodiments, the patient's CDR score decreases from 3 to 2, or from 3 to 1, or from 3 to 0.5, or from 3 to 0, or from 2 to 1, or from 2 to 0.5, or from 2 to 0, or from 1 to 0.5, or from 1 to 0, or from 0.5 to 0. In embodiments, a subject that has been treated with methods described herein shows a slowing of the rate of increase in their CDR score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable CDR score compared to baseline (e.g., prior to treatment or at an earlier timepoint during treatment), which is indicative of a slowing, delay, or cessation of the progression of AD, or a reduction in clinical or cognitive decline.

In embodiments, the one or more identified instruments for assessment of AD, MCI, and/or dementia symptoms is the Frontal Assessment Battery (FAB), which is a screening test that allows an investigator to explore executive functioning through 6 items that include: go/no-go tasks, interference control, verbal fluency, and motor programming. In embodiments, the FAB explores conceptualization, mental flexibility, motor programming, sensitivity to interference, inhibitory control, and environmental autonomy. In embodiments, a cut-off point of ≤11 has been established as an indication of alterations in these cognitive functions.

In embodiments, treatment with the rTMS/gTMS methods described herein result in an improvement (i.e., an increase) in the subject's FAB score compared to a baseline (e.g., prior to treatment or at an earlier timepoint during treatment). In embodiments, the subject's FAB score increases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of decrease in their FAB score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable FAB score compared to a baseline (e.g., prior to treatment or at an earlier timepoint during treatment), which is indicative of a slowing, delay, or cessation of the progression of AD, or a lack of appearance of new clinical, functional, or cognitive symptoms or impairments, or an overall stabilization of disease activity.

In embodiments, the one or more identified instruments for assessment of AD, MCI, and/or dementia symptoms is the verbal fluency test (e.g., phonetic VFT and semantic VFT), for example, as described in Duff et al., 2004 Neurology. 2004; 62:556-562; Ostrosky & Ardila, 1999 J. Int'l. Neuropsyc, Soc'y., 5:413-433, each of which is hereby incorporated by reference in their entirety. In embodiments, the verbal fluency test incorporates a task that asks the subject to generate as many words as quickly as possible according to a criterion, for example using two criteria: semantic (animals) and phonological (letter F). The cut-off points of this task are determined by age and education level.

In embodiments, treatment with the rTMS/gTMS described herein results in an improvement (i.e., an increase) in the subject's verbal fluency test score compared to a baseline (e.g., prior to treatment or at an earlier timepoint during treatment). In embodiments, the subject's verbal fluency test score increases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with the methods described herein shows a slowing of the rate of decrease in their verbal fluency test score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable verbal fluency test score compared to a baseline (e.g., prior to treatment or at an earlier timepoint during treatment), which is indicative of a slowing, delay, or cessation of the progression of AD, or a lack of appearance of new clinical, functional, or cognitive symptoms or impairments, or an overall stabilization of disease activity.

In embodiments, the one or more identified instruments for assessment of AD, MCI, and/or dementia symptoms is the Index of Independence in Daily Life Activities of Katz (ABVD), which evaluates the degree of functional independence in six basic activities (each worth 1 point): bath, clothing, use of the toilet, mobility, continence, and food. In embodiments, a functional dependency is considered in basic activities when at least one of these activities is lost, where a score of 6 points indicates high independence, while a score of 0 indicates high dependence.

In embodiments, treatment with the rTMS/gTMS methods described herein results in an improvement (i.e., an increase) in the subject's ABVD score compared to baseline (e.g., prior to treatment or at an earlier timepoint during treatment). In embodiments, the subject's ABVD score increases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of decrease in their ABVD score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable ABVD score compared to baseline (e.g., prior to treatment or at an earlier timepoint during treatment), which is indicative of a slowing, delay, or cessation of the progression of AD, or a lack of appearance of new clinical, functional, or cognitive symptoms or impairments, or an overall stabilization of disease activity.

In embodiments, the one or more identified instruments for assessment of AD, MCI, and/or dementia symptoms is the Lawton Daily Life Instrumental Activities Scale (AIVD), which rates 8 items with a value of 1 (independent) and 0 (dependent). In embodiments, the final score is the sum of the value of all the answers, it can be between 0 (total dependence) and 8 (total independence), however, an adjustment can be used to reduce score differences due to lifestyle and gender bias, for example eliminating three activities: kitchen, home care, and laundry, resulting in a total of 5 points, where functional independence is considered when all activities are preserved.

In embodiments, a subject that has been treated with rTMS/gTMS methods described herein shows an improvement (i.e., an increase) in the subject's AIVD score compared to a baseline (e.g., prior to treatment or at an earlier timepoint during treatment). In embodiments, the subject's AIVD score increases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of decrease in their AIVD score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable AIVD score compared to a baseline (e.g., prior to treatment or at an earlier timepoint during treatment), which is indicative of a slowing, delay, or cessation of the progression of AD, or a lack of appearance of new clinical, functional, or cognitive symptoms or impairments, or an overall stabilization of disease activity.

In embodiments, the one or more identified instruments for assessment of AD, MCI, and/or dementia symptoms is the Bayer Scale for Daily Life Activities (B-ADL), which is an instrument applied to the informant for the early detection of dementia. In embodiments, the B-ADL includes 25 items, of which 5 measure basic or low cognitive demand activities and the rest more complex activities, where the rating is determined either by the total score or by the index calculated from the total score among the applicable items. In embodiments, the B-ADL specificity and sensitivity values are 0.72 and 0.81, respectively, for the detection of dementia, where a global score for the B-ADL scale is computed by summing across all items without the inclusion of those rated as “unknown” or “not applicable.” In embodiments, the total is then divided by the number of items rated “1” to “10,” resulting in a score that ranges between 1 and 10. In embodiments, two subscores are computed; items 5, 7, and 19 are everyday tasks requiring short-term and long-term memory (subscore 1) and items 21 to 25 relating to cognitive functions important for carrying out everyday life activities (subscore 2). In embodiments, the scores are summed and then divided by the number of relevant items rated “1” to “10,” where a higher score corresponds to more severe deficits.

In embodiments, a subject that has been treated with rTMS/gTMS methods described herein shows an improvement (i.e., a decrease) in the subject's B-ADL score compared to a baseline (e.g., prior to treatment or at an earlier timepoint during treatment). In embodiments, the subject's B-ADL score decreases from 3 to 2, or from 3 to 1, or from 3 to 0.5, or from 3 to 0, or from 2 to 1, or from 2 to 0.5, or from 2 to 0, or from 1 to 0.5, or from 1 to 0, or from 0.5 to 0. In embodiments, a subject that has been treated with methods described herein shows a slowing of the rate of increase in their B-ADL score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable B-ADL score compared to a baseline (e.g., prior to treatment or at an earlier timepoint during treatment), which is indicative of a slowing, delay, or cessation of the progression of AD, or a reduction in clinical or cognitive decline.

In embodiments, the one or more identified instruments for assessment of AD, MCI, and/or dementia symptoms is the Abbreviated geriatric depression scale (GDS-15), which includes 15 questions whose answers are dichotomous. In embodiments, a GDS-15 score greater than or equal to 6 points indicates depression, where internal consistency values of 0.94, test-retest reliability of 0.85, and halving reliability of 0.94 are reported for the GDS-15. In embodiments, convergent validity values of 0.83 have been reported with the Hamilton Depression Assessment Scale and high discrimination in older adults.

In embodiments, a subject treated with rTMS/gTMS methods described herein shows an improvement (i.e., a decrease) in the subject's GDS-15 score compared to a baseline (e.g., prior to treatment or at an earlier timepoint during treatment). In embodiments, the subject's GDS-15 score decreases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of increase in their GDS-15 score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable GDS-15 score compared to a baseline (e.g., prior to treatment or at an earlier timepoint during treatment), which is indicative of a slowing, delay, or cessation of the progression of AD, or a reduction in clinical or cognitive decline.

In embodiments, the one or more identified instruments for assessment of AD, MCI, and/or dementia symptoms is the EuroQol 5-D (EQ-5D), which is a questionnaire composed of three parts that explore the quality of life of subjects. In embodiments, the first part allows the respondent to define the state of health through five dimensions: mobility, personal care, daily activities, pain/discomfort, and anxiety/depression, where each dimension has three levels of severity (no problems, some problems or moderate problems, and more serious problems). In embodiments, the combination of the values of all dimensions generates a 5-digit code that determines the subject's health profile, where a second part is a visual scale graduated from 0 to 100, where the greater the score, the better a perceived state of health. In embodiments, the third part of the EQ-5D collects other data that allow the demographic characterization of the studied group and/or individual, where this scale has shown satisfactory convergent validity compared to other measures of quality of life, as well as adequate discriminant validity. In embodiments, the test-retest reliability is, overall, moderate to good.

In embodiments, a subject treated with rTMS/gTMS methods described herein shows an improvement (i.e., a decrease) in the subject's EQ-5D five-digit score compared to a baseline (e.g., prior to treatment or at an earlier timepoint during treatment). In embodiments, the subject's EQ-5D five-digit score decreases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of increase in their EQ-5D five-digit score by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a stable EQ-5D five-digit score compared to a baseline (e.g., prior to treatment or at an earlier timepoint during treatment), which is indicative of a slowing, delay, or cessation of the progression of AD, or a reduction in clinical or cognitive decline.

In embodiments, the one or more identified instruments for assessment of AD, MCI, and/or dementia symptoms is the Mini-Mental State Examination (MMSE), which provides a 30-point questionnaire that measures cognitive impairment. In embodiments, a MMSE score of 24 or more (out of 30) indicates a normal cognition, whereas scores below 24 indicate severe (<10 points), moderate (10-18 points), or mild (19-23 points) cognitive impairment. In embodiments, the raw score may also need to be corrected for educational attainment and age.

In embodiments, a subject treated with rTMS/gTMS methods described herein shows an improvement (i.e., an increase) in the subject's MMSE score compared to a baseline (e.g., prior to treatment or at an earlier timepoint during treatment). In embodiments, the subject's MMSE score increases by at least 5%, or by at least 10%, or by at least 15%, or by at 20%, or by at least 25%, or by at least 30%, or by at least 35%, or by at least 40%, or by at least 45%, or by at least 50%. In embodiments, a subject that has been treated with methods described herein shows a slowing of the rate of decrease in their MMSE score by at least 5%, or by at least 10%, or by at least 15%, at least 20%, or by at least 25%, or by at least 30%, or by at least 35%, or by at least 40%, or by at least 45%, or by at least 50%. In embodiments, a subject that has been treated with methods described herein shows a stable MMSE score compared to a baseline (e.g., prior to treatment or at an earlier timepoint during treatment), which is indicative of a slowing, delay, or cessation of the progression of AD, or a reduction in clinical or cognitive decline.

In embodiments, the one or more identified instruments for assessment of AD, MCI, and/or dementia symptoms is the Clinical Dementia Rating-Sum of Boxes (CDR-SB) provides a score between 0 and 18, for example as described in O'Bryant et al., 2008, Arch Neurol, Vol. 65: pp. 1091-1095, the entirety of which is hereby incorporated by reference. In embodiments, the CDR-SB score is based on semi-structured interviews of patients and caregiver informants, and yields five degrees of impairment in performance for each of six categories of cognitively-based functioning: memory, orientation, judgment and problem solving, community affairs, home and hobbies, and personal care. In embodiments, each category is scored from 0-3 (e.g., where the five degrees are 0, 0.5, 1, 2, and 3), where the sum of the score for the six categories is the CDR-SB score. In embodiments, the subject for methods herein presents as having a CDR score and/or CDR-SB score of at least about 0.5, at least about or 1, or at least about 2. In embodiments, a decrease in CDR-SB score is indicative of improvement in the subject's condition, whereas an increase in CDR-SB score is indicative of worsening of the subject's condition. In embodiments, a subject that has been treated with methods described herein shows a stable CDR-SB score compared to a baseline (e.g., prior to treatment or at an earlier timepoint during treatment), which is indicative of a slowing, delay, or cessation of the progression of AD, or a lack of appearance of new clinical, functional, or cognitive symptoms or impairments, or an overall stabilization of disease activity.

In embodiments, a subject that has been treated with rTMS/gTMS methods described herein shows an improvement (i.e., a decrease) in the subject's CDR-SB score compared to a baseline (e.g., prior to treatment or at an earlier timepoint during treatment). In embodiments, the subject's CDR-SB score decreases by at least 5%, or by at least 10%, or by at least 15%, or by at least 20%, or by at least 25%, or by at least 30%, or by at least 35%, or by at least 40%, or by at least 45%, or by at least 50%. In embodiments, a subject that has been treated with methods described herein shows a slowing of the rate of increase in their CDR-SB score by at least 15%, or by at least 20%, or by at least 25%, or by at least 30%, or by at least 35%, or by at least 40%, or by at least 45%, or by at least 50%. In embodiments, a subject that has been treated with methods described herein shows a stable CDR-SB score compared to a baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

In embodiments, the one or more identified instruments for assessment of AD, MCI, and/or dementia symptoms is the Repeatable Battery for the Assessment of Neuropsychological Status (RBANS), which includes twelve subtests that are combined to provide five indices, one for each of the five domains tested (e.g., immediate memory, visuospatial/constructional, language, attention, and delayed memory), for example as described in Randolph et al., 1998, J Clin Exp Neuropsychol, Vol. 20: pp. 310-319, the entirety of which is hereby incorporated by reference, where extensive normative values are provided in the testing manuals. In embodiments, an increase in RBANS score is indicative of improvement in the subject's condition, whereas a decrease in RBANS score is indicative of worsening of the subject's condition. In embodiments, a stable RBANS score is indicative of a slowing, delay, or cessation of the progression of AD, or a reduction in clinical or cognitive decline.

In embodiments, a subject that has been treated with rTMS/gTMS methods described herein shows an improvement (i.e., an increase) in the subject's RBANS score compared to a baseline (e.g., prior to treatment or at an earlier timepoint during treatment). In embodiments, the subject's RBANS score increases by at least 5%, or by at least 10%, or by at least 15%, or by at 20%, or by at least 25%, or by at least 30%, or by at least 35%, or by at least 40%, or by at least 45%, or by at least 50%. In embodiments, a subject that has been treated with methods described herein shows a slowing of the rate of decrease in their RBANS score by at least 5%, or by at least 10%, or by at least 15%, at least 20%, or by at least 25%, or by at least 30%, or by at least 35%, or by at least 40%, or by at least 45%, or by at least 50%. In embodiments, a subject that has been treated with methods described herein shows a stable RBANS score compared to a baseline (e.g., prior to treatment or at an earlier timepoint during treatment), which is indicative of a slowing, delay, or cessation of the progression of AD, or a reduction in clinical or cognitive decline.

In embodiments, the methods described herein stimulate neurons around about 1 cm to about 4 cm from the skull, about 2 cm to about 4 cm from the skull, about 1 cm to about 3 cm from the skull, or about 2 cm to about 3 cm from the skull.

In embodiments, the method stimulates alterations in gamma, theta, alpha, and/or beta oscillation in the subject's brain. In embodiments, “oscillation” and “oscillation power,” are used interchangeably. In embodiments, the method stimulates alterations in gamma oscillation in the subject's brain. In embodiments, the method stimulates alterations in theta oscillation in the subject's brain.

In embodiments, the method stimulates neurons throughout the subject's brain. In embodiments, the method substantially stimulates neurons in the left hemisphere of the subject's brain. In embodiments, the method substantially stimulates neurons in the frontal lobe of the subject's brain. In embodiments, the method substantially stimulates neurons in the cerebral cortex of the patient. In embodiments, the method stimulates neurons outside of the patient's left prefrontal dorsolateral cortex. In embodiments, “substantially stimulate,” refers to a degree or level of stimulation that exceeds that of a subject not receiving the rTMS/gTMS methods herein, while participating in the same activities (e.g., seated with eyes open, seated with eyes closed, reading, concentrating, performing one or more evaluations of Table 1, etc.).

In embodiments, the methods herein result in cognitive improvement without affecting amyloid plaques and neurofibrillary tangles present in the subject's brain. That is, in embodiments, the present methods are effective in the absence of amyloid-mediated effects, or in the absence of proteinaceous inclusions in the brain.

In embodiments, the methods herein prevent or delay the progression of MCI to AD. In embodiments, the treatment method improves and/or prevents diminution of cognitive traits of the subject. For example, methods of the present disclosure provide for slowing memory loss and/or retaining and/or increasing memory capacity, memory function, and/or cognitive function in the subject having MCI or AD. In embodiments, methods herein include treating psychiatric symptoms linked to MCI and AD, including, but not limited to, depression, anxiety, appetite fluctuation, and issues with sleep.

In embodiments, prevention or delay of the progression of MCI to AD, as well as one or more symptoms associated therewith (e.g., depression, anxiety, appetite fluctuation, sleep troubles, etc.) are assessed using one or more of the instruments for assessment of MCI/AD symptoms, e.g., as listed in Table 1.

In embodiments, methods herein result in an increase in one or more of a F-NAME score, a RAVLT score, a pVFT score, a sVFT score following administration of the rTMS/gTMS, which indicates one or more of increased memory capacity, memory function, or cognitive function in the subject.

In embodiments, methods herein result in an increase in a MoCA score and/or a decrease in an ADS-Cog score following administration of the rTMS/gTMS, which indicates one or more of increased memory capacity, memory function, or cognitive function in the subject. In embodiments, the method increases the subject's MoCA score, as compared to a MoCA score at baseline. In embodiments, the treatment decreases the subject's ADAS-COG score, as compared to an ADAS-COG score at baseline.

In embodiments, methods herein result in an improvement of one or more scores used to assess MCI, AD, and/or dementia, e.g., as described in Table 1.

Methods herein, in embodiments, are substantially free of adverse effects. In embodiments, adverse effects include epileptic seizures, nausea, headache, vagal response, musculoskeletal pain, scalp petechial rash, scalp pain, edema, and bruising.

Methods herein, in embodiments, obviate the need for one or more treatments. In embodiments, the subject's response to the methods herein results in a decrease in the frequency and/or dosage of one or more treatments.

### Additional Therapeutic Agents, Treatments, and Combination Therapy

In embodiments, methods herein include administering one or more additional therapeutic agents and/or treatments. For example, in embodiments, methods herein represent a treatment which is part of a regimen, intended to be undertaken as part of a suite of different categories of treatments.

In embodiments, methods herein include administering one or more treatments as a function of the subject's response to applying the rTMS/gTMS. In embodiments, the subject's response to the rTMS/gTMS indicates a change in one or more symptoms of the AD, MCI, and/or dementia, where the change informs the administration. In embodiments, this informed administration includes discontinuing the administration of one or more treatments. In embodiments, the administration of the treatment is in response to an assessment of the subject's response to the rTMS/gTMS, as measured and/or assessed by one or more methods, tests, and/or scales listed in Table 1. In embodiments, the one or more treatments is administered prior to applying the rTMS/gTMS, substantially simultaneously with applying the rTMS/gTMS, or after applying the rTMS/gTMS. In embodiments, the one or more treatments are for AD, MCI, and/or dementia.

In embodiments, the one or more treatments comprises a pharmacological treatment, psychosocial treatment, caregiving treatment, diet-based treatment, and/or exercise.

In embodiments, the pharmacological treatment (e.g., additional therapeutic agent) comprises one or more therapeutic agents for AD, MCI, and/or dementia. In embodiments, the one or more therapeutic agents for AD, MCI, and/or dementia comprises a neurological drug, corticosteroid, antibiotic, antiviral agent, anti-Tau antibody, Tau inhibitor, anti-amyloid beta antibody, beta-amyloid aggregation inhibitor, anti-BACE1 antibody, BACE1 inhibitor, acetylcholinesterase inhibitor (tacrine, rivastigmine, galantamine, and donepezil), NMDA receptor antagonist (memantine), gene therapy (AAVs), vaccine-based immunotherapy agent (AB peptide and/or nucleic acid encoding Aβ peptide), antibody-based agent (bapineuzumab, solanezumab, crenezumab, BAN2401, aducanumab, ALZ801); and/or a liposome, micelle, and/or metal nanoparticle-based drug delivery agent. In embodiments, the additional therapeutic agent (e.g., pharmacological treatment) is a treatment for AD, including, but not limited to, donepezil (ARICEPT), rivastigmine (EXELON), galantamine (RAZADYNE), and memantine (NAMENDA).

In embodiments, the psychosocial treatment comprises speech therapy, behavioral therapy, cognitive therapy, cognitive/memory training, reminiscence therapy, validation therapy, supportive psychotherapy, sensory integration (Snoezelen), simulated presence therapy, and/or pet therapy.

In embodiments, the caregiving therapy comprises hospice care, feeding tubes, safety locks, and/or movement aids.

In embodiments, the diet-based treatment comprises liquid diets, pureed foods, and/or diets for difficulty with swallowing.

In embodiments, the one or more additional therapeutic agents is administered prior to applying the rTMS/gTMS, substantially simultaneously with applying the rTMS/gTMS, or after applying the rTMS/gTMS. In embodiments, “substantially simultaneously,” as used herein, refers to the administration of one or more therapeutic agents and/or treatments with the rTMS/gTMS methods herein with a time of separation of no more than about 60 minutes, such as no more than about 30 minutes, no more than about 20 minutes, no more than about 10 minutes, no more than about 5 minutes, or no more than about 1 minute. In embodiments, the additional therapeutic agent and the methods herein are administered to a subject simultaneously, but the release of the additional therapeutic agent from its respective dosage form and the methods herein occur sequentially.

In embodiments, co-administration does not require the additional therapeutic agent and the TMS/gTMS to be administered simultaneously, if the timing of their administration is such that the pharmacological activities of the additional therapeutic agent and the methods herein overlap in time. For example, in embodiments, the additional therapeutic agent and the rTMS/gTMS can be administered sequentially. In embodiments, the term “sequentially,” as used herein means that the additional therapeutic agent and the rTMS/gTMS are administered with a time separation of more than about 60 minutes. For example, the time between the sequential administration of the additional therapeutic agent and the administration of rTMS/gTMS can be more than about 60 minutes, more than about 2 hours, more than about 5 hours, more than about 10 hours, more than about 1 day, more than about 2 days, more than about 3 days, or more than about 1 week apart. In embodiments, the administration times depend on the rates of metabolism, excretion, and/or the pharmacodynamic activity of the additional therapeutic agent being administered. In embodiments, either the additional therapeutic agent or the rTMS/gTMS are administered first.

### Subjects and Subject Selection

In embodiments, the subject of methods herein is human.

In embodiments, the human is an adult human. In embodiments, the human is a geriatric human. In embodiments, the human is referred to as a patient and/or is a person diagnosed with, presenting with, and/or suspected of having Alzheimer's disease (AD), mild cognitive impairment (MCI), dementia, and/or neurodegenerative disease, generally.

In embodiments, the human subject exhibits one or more biomarkers for AD, MCI, and/or dementia, including but not limited to, high AB in cerebrospinal fluid, high Tau in cerebrospinal fluid, and the presence of the ApoE4 allele. In embodiments, the human subject exhibits one or more diagnostic outcomes related to AD, MCI, and/or dementia, including for example having a positive florbetapir PET scan, or having a positive Tau PET scan.

In embodiments, the human subject has been prescribed and/or is undergoing treatment with one or more of a neurological drug, corticosteroid, antibiotic, antiviral agent, anti-Tau antibody, Tau inhibitor, anti-amyloid beta antibody, beta-amyloid aggregation inhibitor, anti-BACE1 antibody, BACE1 inhibitor, acetylcholinesterase inhibitor (tacrine, rivastigmine, galantamine, and donepezil), NMDA receptor antagonist (memantine), gene therapy (AAVs), vaccine-based immunotherapy agent (AB peptide and/or nucleic acid encoding Aβ peptide), antibody-based agent (bapineuzumab, solanezumab, crenezumab, BAN2401, aducanumab, ALZ801); and/or a liposome, micelle, and/or metal nanoparticle-based drug delivery agent. In embodiments, the additional therapeutic agent (e.g., pharmacological treatment) is a treatment for AD, including, but not limited to, donepezil (ARICEPT), rivastigmine (EXELON), galantamine (RAZADYNE), and memantine (NAMENDA).

In embodiments, the human subject is undergoing psychosocial treatment, including for example, speech therapy, behavioral therapy, cognitive therapy, cognitive/memory training, reminiscence therapy, validation therapy, supportive psychotherapy, sensory integration (Snoezelen), simulated presence therapy, and/or pet therapy.

In embodiments, the human subject is undergoing caregiving therapy, including hospice care, feeding tubes, safety locks, and/or movement aids.

In embodiments, the human subject is undergoing a diet-based treatment, including liquid diets, pureed foods, and/or diets intended for individuals experiencing difficulty swallowing.

In embodiments, the human subject exhibits a score from one or more assessments relating to one or more categories of: Cognitive Impairment, Activities of Daily Living, Global Impression, Behavior-Neuropsychology, Quality of Life, Communication/Social, Interaction, and Multi-Domain, which indicates AD, MCI, and/or dementia.

In embodiments, the human exhibits a score from one or more assessments which indicates AD, MCI, and/or dementia, e.g., as listed in Table 1, including the ADAS-Cog, BIMC, CAMCOG, F-NAME, MDRS, MMSE, NTB, pVFT, RAVLT, SVFT, SCIP, SIB, SKT, ADL-P, ADLQ, ADCS-ADL19/23, B-ADI, Bristol ADL, BANS-S, Barthel ADL, DAD, GERRI, IDDD, Katz ADL, PGDRS, RDRS, PSMS, SMAF, Weintraub ADL, ADCS-CGIC, CDR, CGI, CIBI, FAST, GDS, HDR, NYU CIBIC-Plus, AMS, IA, BEAM-D, BEHAVE-AD, BPRS, CERAD-BRSD, CMAI, CSDD, DBD, DMAS, GDS, HoNOS 65+, NPI, NRS, RMBPC, ADRQL, DQOL, DS-DAT, OERS, PDS, QOL-AD, QUALID, CLB, CPS, ERT, MOSES, PKT, PPQSA, SCS, BGP, GBS, NOSGER, SCAG, VL.

In embodiments, the human has an age in a range of from about 18 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

### Kits

In aspects, described herein are kits comprising the rTMS/gTMS device which can find use in performing methods described herein.

In embodiments, the components of the present disclosure are assembled into a kit. In embodiments, the kit includes a rTMS/gTMS device and/or one or more therapeutics agents. In embodiment, the rTMS/gTMS device is a wearable device which is suitable for being self-applied and/or used in a home setting or outpatient setting.

In embodiments, the kit can include one or more containers housing components for performing the methods described herein, and optionally instructions for use. In embodiments, the kit can further include components useful for performing the methods herein. Each component of the kits, where applicable, can include therapeutic agents provided in liquid form (e.g., in solution) or in solid form, (e.g., a dry powder). In embodiments, one or more of the components are reconstitutable or otherwise processible (e.g., to an active form), for example, by the addition of a suitable solvent or other species (for example, water, saline, dextran, or PBS), which may or may not be provided with the kit.

In embodiments, the kits can optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions can be in a form prescribed by a governmental agency regulating the manufacture, use, or sale of pharmaceuticals or biological products, which can also reflect approval by the agency of manufacture, use, or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits can include other components depending on the specific application, as described herein.

In embodiments, the kits can contain any one or more of the components described herein in one or more containers. The components can be prepared sterilely, packaged (e.g., in a syringe), and shipped refrigerated. Alternatively, components can be housed in a vial or other container for storage. A second container can have other components prepared sterilely. Alternatively, the kits can include the active agents premixed and shipped in a vial, tube, or other container.

In embodiments, the kits can include a variety of forms, e.g., for therapeutic agents, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits can be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits can also include other components, depending on the specific application, for example, containers, salts, buffers, reagents, syringes, needles, a fabric, such as gauze, for applying or removing a disinfecting agent, disposable gloves, a support for the agents prior to administration, etc.

Without further elaboration, it is believed that one skilled in the art can, based on the above description, utilize the present disclosure to its fullest extent. The following specific embodiments are, therefore, to be construed as merely illustrative, and not limiting of the remainder of the disclosure in any way whatsoever.

### EXAMPLES

The examples herein are provided to illustrate advantages and/or benefits of the disclosure and to further assist a person of ordinary skill in the art with making and using the devices and methods disclosed herein. The examples herein are also presented to more fully illustrate aspects of the present embodiments. The examples should in no way be construed as limiting the scope of the present disclosure, as defined by the appended claims. The examples include or incorporate any of the variations, aspects, or embodiments as described herein. The variations, aspects, or disclosure described herein may also further each include or incorporate the variations of any or all other variations, aspects, or embodiments described herein.

### Example 1: Low-Intensity Transcranial Magnetic Stimulation at Gamma Frequencies Safely Modulates Gamma, Theta, and Alpha Oscillations in Individuals with Mild Alzheimer's Disease Dementia: A Randomized Crossover Pilot Study

This Example summarizes a randomized, double-blind, sham-controlled, crossover human pilot study for evaluating low-intensity gamma magnetic stimulation (rTMS/gTMS) in participants with AD and/or presenting with dementia. The study was designed to evaluate the safety of physiologically relevant frequency rTMS/gTMS for feasibility of long-term, or chronic, use to initiate long-term improvements in cognition. Significant changes in cognition were not anticipated from the single gTMS session. Participants were randomized in a 1:1 ratio, bifurcated based on beginning intervention. Each participant received both a single 40-minute session of real gamma-frequency repetitive transcranial magnetic stimulation (gTMS), and a sham stimulation session of the same duration. A washout period of one week was used between crossover of the intervention to prevent potential carryover effects. To ensure blinding, researchers conducting all clinical evaluations remained unaware of the intervention administered to each participant, while a separate researcher administered either the real or sham stimulation. Participants were also blinded throughout the study, where the physiologically relevant frequencies of the magnetic stimulation employed did not elicit any sensory cues that potentially compromised the blinding integrity from the participants' perspective.

### Methods:

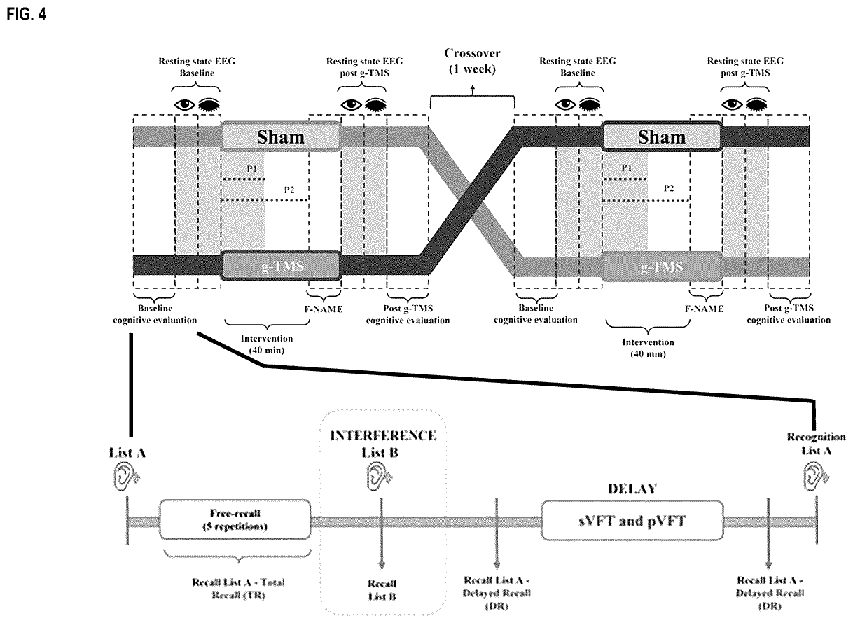

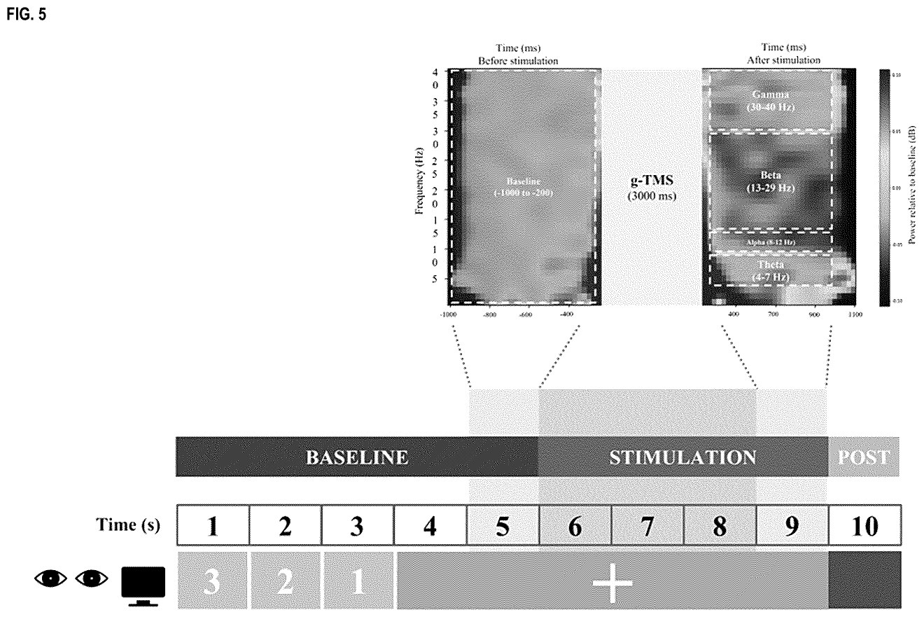

Each session consisted of a baseline and post-stimulation cognitive assessment, along with recordings of electroencephalogram (EEG) activity during periods with eyes open and eyes closed. The stimulation session employed two distinct paradigms designed to assess different aspects of gTMS effects. The first paradigm (e.g., denoted as “P1,” in FIG. 4 and FIG. 5) included applying 110 bursts of 40 Hz over a 20 min period. The time period after each burst was used to perform time-frequency analyses. This enabled examination of the immediate after-effects and short-term electrophysiological changes induced by gTMS. The second paradigm (e.g., denoted as “P2,” in FIG. 4) involved a continuous 40 Hz stimulation lasting for a period of 40 min. This paradigm investigated the sustained effects of gTMS over a longer duration and provided insights into potential long-lasting changes in electrophysiological activity. At the end of P2, a cross-modal associative memory test, known as the Face-Name Associative Memory Test (F-NAME test), was administered (e.g., as denoted in FIG. 4).

Sample Size Calculation: The sample size for the current study was determined, for example as described in Benussi A, et al., “Exposure to gamma tACS in Alzheimer's disease: A randomized, double-blind, sham-controlled, crossover, pilot study,” Brain Stimul, Vol. 14, 2021: pp. 531-40, the entirety of which is hereby incorporated by reference. Benussi et al., reported that the mean and standard deviation (SD) of the Rey Auditory Verbal Learning Test (RAVLT) score for the sham session as 19.4 and 6.8, respectively, with an alpha significance level of 0.05 and a statistical power of 0.8 were employed for calculations. The minimum number of subjects required for a two-way hypothesis was calculated to be 18 subjects per group. To account for the potential non-parametric distribution of the data, this value was then multiplied by 1.2, resulting in 21.6 subjects. Taking into consideration the potential loss of 20% in the sample, the initial sample size of 21.6 subjects was further adjusted. As a result, the study involved the recruitment of a total of 27 participants:

$$N = \frac{n}{1 - percentage of expected losses} = \frac{21.6}{( 1 - 0.2 )} = 27$$

Participants: Participants for the trial were identified and evaluated during their consultations at a memory clinic. Candidates were aged 65 years or older and exhibit confirmed diagnosis of mild dementia due to Alzheimer's Disease (AD). Diagnosis was ascertained by a collaborative assessment between a consulting geriatrician/neurologist and a neuropsychologist, in accordance with the diagnostic standards delineated in the Diagnostic and Statistical Manual of Mental Disorders, Fifth Edition (DSM-5) and the criteria stipulated by the National Institute of Neurological and Communicative Disorders and Stroke-Alzheimer's Disease and Related Disorders Association (NINCDS-ADRDA). Subjects were not included in the study if they had uncontrolled medical conditions, a diagnosis of major depressive disorder, metallic implants, a history of seizures, or previous use of any brain stimulation devices. Candidates who were undergoing pharmacological treatment were eligible to participate, provided that their medication regimen had remained consistent for at least 12 weeks prior to the start of the intervention. However, the introduction of new medications and/or cognitive interventions after the first session of intervention was not permitted.

Intervention: The device operated by delivering electric current through a coil to generate a rapidly changing magnetic field at a gamma frequency of 40 Hz. For this study, a custom-made circular coil with a diameter of 50 mm was employed. The coil was positioned beneath an EEG flexible cap and featured a central hole that accommodated the Pz electrode, encompassing both the medial parietal cortex and the precuneus. This location was selected based on previous positive cognitive outcomes through electrical stimulation in this area (Benussi et al., 2021). The magnetic pulses were administered with an approximate magnitude of 10 mT. Modeling software was employed to accurately calculate the magnetic field intensity to induce an electric field of 1 V/m² focused at a distance of 3 cm into the brain from the coil. During each session, the intervention consisted of two different paradigms, e.g., as described in FIG. 4. The first paradigm allowed for the evaluation of immediate after-effects and short-term electrophysiological changes resulting from gamma magnetic stimulation. Upon completion of the first paradigm, the second paradigm automatically commenced, involving continuous 40 Hz stimulation for 20 minutes without interruption.

EEG Data Acquisition: EEG data was acquired using an EMOTIV EPOC Flex EEG system (EMOTIV, San Francisco, USA), equipped with 32 saline electrodes. These electrodes were affixed to participants' heads using a flexible cap (EASYCAP, Herrsching, Germany), and their locations were configured based on the international 10-20 system (Herwig et al., 2003). The impedance of the electrodes was consistently maintained below 20 kΩ throughout all experimental phases. The EEG data was sampled at a rate of 1,024 Hz, initially filtered within the range of 0.2 Hz to 45 Hz, and subsequently downsampled to 128 Hz. All EEG data acquisition procedures were performed using the EMOTIV PRO software (EMOTIV, San Francisco, USA). For each session, EEG recordings were conducted at three distinct time points: 1) prior to stimulation (2.5 min, eyes open; 2.5 min eyes closed), 2) during the first 20 minutes of stimulation, and 3) immediately after stimulation (2.5 min, eyes open; 2.5 min, eyes closed) (e.g., as shown in FIG. 5). To accurately timestamp the initiation of stimulation during the EEG recording, an external trigger (EMOTIV Extender) was synchronized with the stimulator, which marked the events at the start of the intervention.

EEG Data Pre-Processing: The open-source MNE-Python package for analysis of human neurophysiological data, along with the NumPy and Matplotlib libraries were used for the data preprocessing (Gramfort et al., 2013; Harris et al., 2020; Hunter et al., 2007). During the first 20 minutes of stimulation, the raw EEG data was fitted to an Independent Component Analysis (ICA) model consisting of 31 components. The resulting components were visually inspected as time-series and spatial maps to identify and exclude components representing eye-blinking and cardiac activity artifacts. Furthermore, all 32 channels (of each electrode) were examined for excessive noise or artifacts, and if any were present, the channels were interpolated. The data was then subjected to a high-pass filter (1 Hz) and a low-pass filter (45 Hz), followed by re-referencing to the common average. To eliminate artifacts caused by the TMS pulses, a specific time window was excised from the data, comprising 200 ms before stimulation, 3000 ms during stimulation, and 200 ms after stimulation. Two distinct epochs were then created and concatenated for subsequent analysis: a baseline epoch, spanning from 1000 ms before stimulation to 200 ms before stimulation, and a period of interest epoch, encompassing data from 200 ms after stimulation to 1000 ms after stimulation (e.g., as shown in FIG. 5). Another round of ICA was performed on the concatenated epochs, this time with 23 components, to identify and remove components containing artifacts resulting from the concatenation of epochs. Finally, all epochs were manually inspected to identify and discard any remaining epochs of poor quality. The epoched data was processed for time-frequency analysis.

For the baseline and post-stimulation EEG recordings during conditions where the participants' eyes were open and eyes were closed, the EEG data was imported and preprocessed using a similar approach. An ICA model consisting of 31 components was fitted to identify eye-blinking and cardiac activity artifacts in the raw data. All 32 channels (for each electrode) were examined for excessive noise or artifacts, and if necessary, the channels were interpolated. The data was high-pass filtered (1 Hz), low-pass filtered (45 Hz), and re-referenced to the common average. A new set of events was created, with events occurring every 5 seconds, then data was epoched accordingly, ranging from −1.5 to +1.5 seconds relative to the event onset. The epoched data was visually inspected to identify and mark any low-quality epochs for exclusion. Subsequently, the epoched data was processed for spectral analysis.

Data Analysis—Baseline and post-stimulation EEG: The preprocessed epochs from real and sham conditions were used to calculate the time frequency representation of each epoch using Morlet wavelets. The number of cycles per wavelength was one half of the analyzed frequency (1 to 40 Hz). For each subject, the average time frequency representation was calculated and then these individual averages were used to create a grand average for each condition. To evaluate differences between conditions, regions of interest in the time frequency plane were selected beforehand. Four different regions of interest (ROI) were defined for each electrode depending on the time of interest (between 500 ms after stimulation and 900 ms after stimulation) and the activity in theta (4-8 Hz), alpha (9-12 Hz), beta (13-30 Hz), and gamma (31-40 Hz.) frequency bands. All values were normalized using a log ratio method to baseline (the period between 900 and 500 before stimulation). For each of the 32 electrodes, the average activity of each of the 4 ROI was calculated and then compared between conditions.

Data Analysis—Baseline and post-resting state EEG: For each preprocessed epoch of both conditions (sham and real) at both time points (baseline and post-stimulation) power spectral density was computed (for example, as described in Slepian D. “Prolate Spheroidal Wave Functions, Fourier Analysis, and Uncertainty—V: The Discrete Case,” Bell System Technical Journal, Vol. 57, 1978: pp. 1371-430, the entirety of which is hereby incorporated by reference). Individual PSD representations of epochs were then averaged within subjects and then a grand average for each condition was created. The power activity of the grand average was calculated in the canonical frequencies mentioned above to compare between conditions the power activity post intervention was normalized using a log ratio method to the baseline power activity (baseline recording).

Statistical Analysis of EEG Analysis: To evaluate the degree of change in the power activity immediately post-stimulation compared to baseline, a Wilcoxon signed-rank test was performed between conditions for all electrodes at each of the four ROI frequency bands. The same approach was used to compare between baseline and post-resting state stimulation recordings (eyes open and closed). Due to the high number of comparisons, a p value correction for multiple comparison using False Detection Rate (FDR) was used (for example, as described in Genovese C R, et al. “Thresholding of Statistical Maps in Functional Neuroimaging Using the False Discovery Rate,” NeuroImage, Vol. 15, No. 4, 2002: pp. 870-8, the entirety of which is hereby incorporated by reference).

Cognitive Assessment—Rey Auditory Verbal Learning Test (RAVLT): To evaluate acute changes in cognition secondary to gamma stimulation, a baseline and a post-stimulation Rey Auditory Verbal Learning Test (RAVLT), as well as semantic and phonetic Verbal Fluency Test (SVFT & pVFT, respectively) were performed (e.g., as shown in FIG. 4). The RAVLT evaluated the verbal episodic memory and was designed as a list-learning paradigm (for example, as described in Bean J. “Rey Auditory Verbal Learning Test, Rey AVLT,” In: Kreutzer J S, DeLuca J, Caplan B, editors. Encyclopedia of Clinical Neuropsychology, New York, NY: Springer; 2011, p. 2174-5, the entirety of which is hereby incorporated by reference). The application of the RAVLT included the subject hearing a list of 15 different nouns (List A) and being asked to recall as many words as possible after each of 5 repetitions of free-recall. This was followed by an interference trial, where the subject heard a second list of nouns (interference List B) and was asked to recall as many as possible. Total recall (TR) was then assessed by asking the subject to recall as many words as possible from List A. A TR score was obtained by adding all recalled words from the 5 free-recall repetitions. To avoid learning effects, different lists of words were used for the baseline and post-stimulation evaluation.

Cognitive Assessment—Verbal Fluency Tests (VFT): To evaluate acute changes in cognition secondary to gamma stimulation, a baseline and a post-stimulation VFT assessments were performed using short tests that assess the ability to retrieve specific information within restricted search parameters (for example, as described in Patterson J. “Verbal Fluency,” In: Kreutzer J S, DeLuca J, Caplan B, editors. Encyclopedia of Clinical Neuropsychology, New York, NY: Springer; 2011, p. 2603-6, the entirety of which is hereby incorporated by reference). Both semantic (SVFT) and phonemic (pVFT) tests were applied to evaluate the effect of the intervention on information retrieval from memory through verbal fluency. During the delay period of the RAVLT, participants were provided 1 minute to generate a list of words that shared a semantic feature (e.g., animals or fruits), and provided a second 1-minute period to generate a list of words that began with a single letter (e.g., “F” or “L”). The first test evaluated semantic fluency, while the second assessed phonemic fluency. The total number of words for each test was used to assess verbal fluency before and after sham or real stimulation.

Cognitive Assessment—Face-Name (F-NAME) association task: The F-NAME association task was performed to examine the impact of gTMS on episodic associative memory (e.g., as shown in FIG. 4). The task included three phases: practice, encoding, and retrieval. During the practice phase, participants were given instructions for the test along with one example. During the encoding phase, participants were presented with 12 unfamiliar faces along with their corresponding names. They were then required to determine whether each given name suited the respective face. Once this encoding phase was completed, participants proceeded to the retrieval phase, which encompassed three tests. In the first test, participants were shown three different faces without any associated names. Among these faces, one belonged to the initial set of 12 faces, while the other two were new faces. The participants' were tasked with selecting the face they remembered from the encoding phase. For the second test, participants were presented with a single face and were asked to recall the first letter of the name associated with that face. Lastly, the third test involved participants being shown a face alongside three different names, including the correct name and two incorrect names. The participants' objective was to choose the accurate name corresponding to the displayed face. To ensure clarity and comprehension, the researcher verbally explained the instructions and guided the participants through the practice and the encoding and retrieval phases of the test.

Statistical Analysis of Clinical and Cognitive Data: Statistical analyses were performed using a One-way ANCOVA conducted to assess the effect of gTMS on RAVLT, sVFT, and pVFT clinical scores post-intervention whilst controlling for baseline scores. Paired T-tests were used to assess differences in the F-NAME test scores (face, letter, name, and total) between sham and gTMS during stimulation.

### Results:

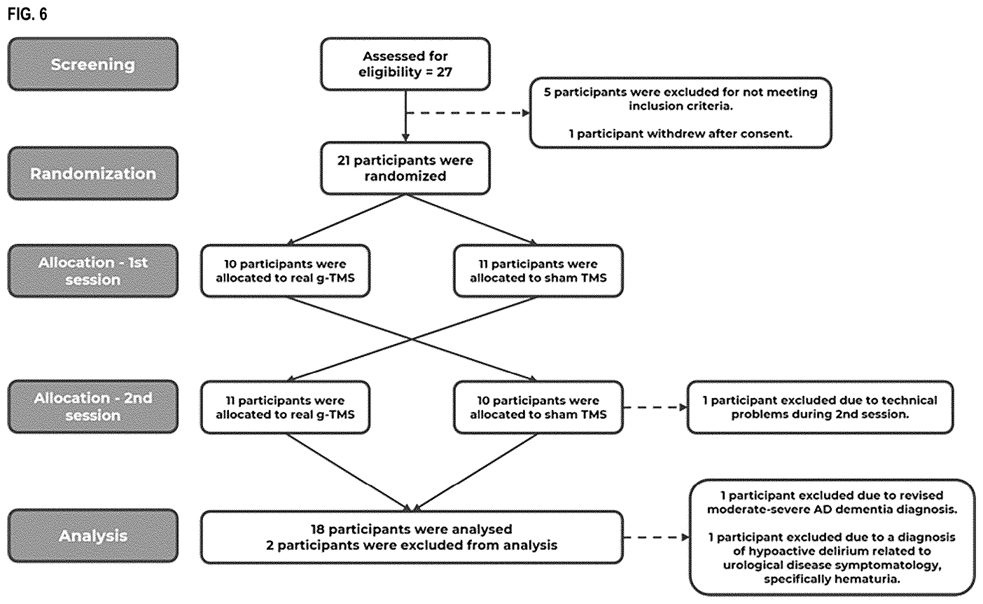

Twenty-seven participants were initially screened, with 5 participants not meeting the inclusion criteria and 1 consented but withdrawn from study prior to randomization. Twenty-one participants with mild AD dementia were enrolled and randomized to receive gTMS or sham during the first session in a 1:1 ratio. Eighteen participants completed the study and were included in the final analysis (e.g., as shown in FIG. 6). Demographic and clinical information for included participants is presented in Table 2.

TABLE 2
Demographic and clinical information for included participants.

| Variable | Values (n = 18) |
|---|---|
| Age, years | 76.7 ± 5.7 |
| Sex, female | 44% |
| Education, years | 13.4 ± 6.7 |
| MoCA Total Score | 16.1 ± 5.9 |
| ADAS-Cog Total Score | 26.4 ± 7.7 |
| Katz ADL Index | 5.9 ± 0.5 |
| CDR Score | 0.9 ± 0.2 |
| GDS Score | 2.6 ± 2.5 |

MoCA = Montreal Cognitive Assessment; ADAS-Cog = Alzheimer's Disease Assessment Scale-Cognitive Subscale; KATZ ADL = Katz Index of Independence in Activities of Daily Living; CDR = Clinical Dementia Rating Scale; GDS = Geriatric Depression Scale.
All values, except for sex, are expressed as mean ± SD.

Out of the 18 participants involved in the intervention, 16 experienced no adverse effects. However, one participant reported a headache one day after the first session (sham), with no apparent triggers or factors that could have worsened the condition. To alleviate the symptoms, the participant was prescribed a non-steroidal anti-inflammatory drug (NSAID) and showed an improvement in symptoms during the follow-up period. Another participant reported eye pain at the conclusion of both the first (sham) and second (gTMS) sessions. It was determined that the eye pain was not associated with the intervention itself, but rather with a pre-existing condition. Additionally, this same participant experienced a headache after the first session, as well as headache and nausea following the second session. While the symptoms of the first case were alleviated after taking oral pain relief medication, the symptoms persisted for a period of two weeks after the second session. Overall, the intervention was well-tolerated by the majority of participants, with only a few cases of minor and unrelated adverse effects reported.

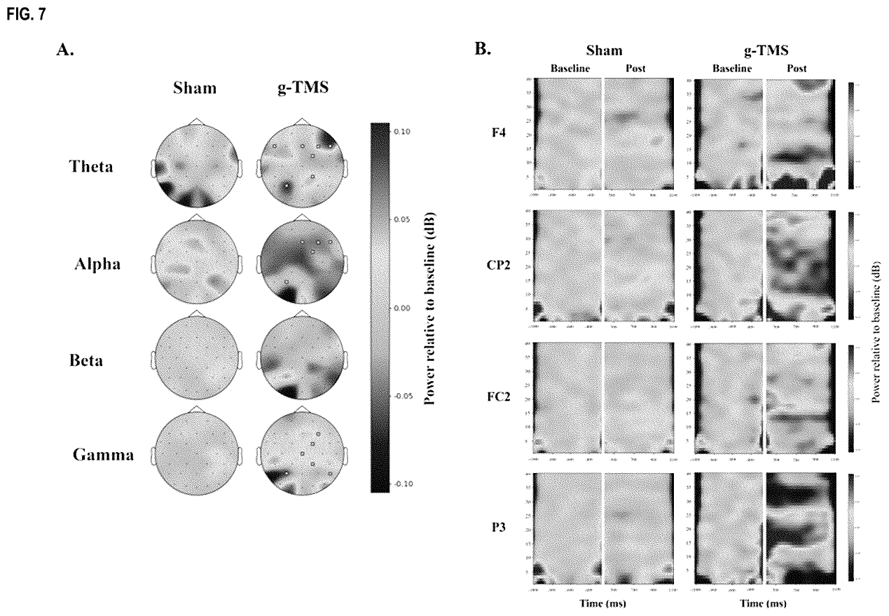

The results demonstrated, inter alia, that gTMS induced transient changes in gamma, theta, and alpha oscillations during stimulation in patients with mild AD and/or dementia. Significant changes in gamma, theta, and alpha oscillation power were observed in EEG recordings immediately after gTMS compared to sham stimulation, e.g., as shown in FIGS. 7A-7B, mainly located at the frontal and parietal regions of the brain. Specifically, the gTMS group exhibited a significant increase in gamma power at the frontal and parietal electrodes F4 (p=0.002, q=0.02), FC2 (p<0.0001, q<0.001), Cz (p=0.002, q=0.02), CP2 (p=0.005, q=0.03), and P8 (p=0.004, q=0.03), while a significant decrease was observed at P3 (p=0.002, q=0.02). Similar patterns were found for theta power, with significant changes observed at F4 (p=0.003, q=0.03), FC2 (p=0.002, q=0.02), CP2 (p=0.002, q=0.02), and P3 (p<0.0001, q=0.002) electrodes, as well as at F7 (p=0.005, q=0.03), Fz (p<0.001, q=0.02), and F8 (p=0.0001, q=0.005) electrodes where theta power was increased compared to sham stimulation. Changes in alpha indicated decreased power at Fz (p=0.002, q=0.02), F4 (p=0.0005, q=0.02), F8 (p=0.002, q=0.02), FC2 (p=0.001, q=0.02), and P3 (p=0.003, q=0.03) electrodes.

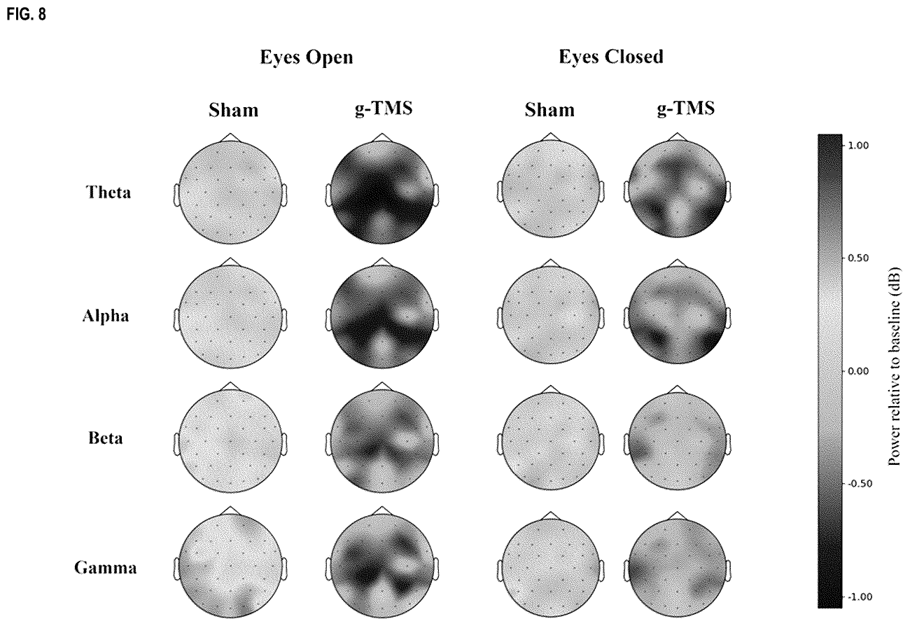

To investigate the persistence of these effects following stimulation, EEG resting state recordings were conducted before (baseline) and after (post) gTMS or sham stimulation, with both eyes open and closed. The analysis revealed an overall decrease in power after gTMS across all frequency ranges, e.g., as seen in FIG. 8.

An analysis using one-way ANCOVA was conducted to examine the impact of gTMS on cognition, focusing on the RAVL and sVFT/pVFT test scores after stimulation, while adjusting for baseline scores. The results revealed no significant improvement in the RAVL [F_{(1,33)}=0.027, p=0.871] and sVFT/pVFT [F_{(1,33)}=0.165, p=0.687; F_{(1,33)}=0.154, p=0.697] performance following a single gTMS stimulation compared to the sham condition (e.g., as shown in Table 3). The analysis of the F-NAME association task using a paired-T test produced similar outcomes, indicating that there were no significant changes in the final scores observed during gTMS compared to sham (e.g., as shown in Table 3).

TABLE 3
Cognitive assessment scores.

| | | Post | | | | | |
|---|---|---|---|---|---|---|---|
| | | Sham | | gTMS | | | |
| | | | Adjusted | | Adjusted | | |
| | | Mean ± | Mean ± | Mean ± | Mean ± | F test | |
| | | SD | SEM | SD | SEM | statistic | p-value |
| RAVL | Total | 22.6 ± 9.1 | 22.4 ± 1.3 | 21.9 ± 7.9 | 22.1 ± 1.3 | 0.027 | 0.871 |
| sVFT | recall | 12.2 ± 5.5 | 12.0 ± 1.1 | 12.4 ± 4.8 | 12.6 ± 1.1 | 0.165 | 0.687 |
| pVFT | | 9.3 ± 4.2 | 9.3 ± 0.6 | 8.9 ± 3.9 | 8.9 ± 0.6 | 0.154 | 0.697 |
| F- | Face | 9.6 ± 2.2 | | 9.9 ± 2.2 | | | 0.341 |
| NAME* | Letter | 1.9 ± 1.9 | | 2.1 ± 1.9 | | | 0.733 |
| | Name | 6.1 ± 1.2 | | 6.3 ± 1.7 | | | 0.641 |
| | Total | 17.6 ± 3.8 | | 18.3 ± 4.4 | | | 0.339 |

gTMS = gamma repetitive Transcranial Magnetic Stimulation; RAVL = Rey Auditory Verbal Learning test; sVFT = semantic Verbal Fluency Test; pVFT = phonemic Verbal Fluency Test; F-NAME = face-name associations task; SD = Standard Deviation; SEM = Standard Error of the Mean.

The changes in EEG recordings were evaluated immediately after stimulation as well as during post-stimulation resting state. The results revealed, inter alia, that a single session of low-intensity gTMS targeting the precuneus led to electrophysiological changes in both nearby and distant cortical areas, primarily in the gamma and theta frequency ranges. While small improvements in F-NAME scores were observed, the changes were not statistically significant, which is expected for a single session of gTMS intervention. Of the 18 participants, 16 tolerated the gTMS intervention well, and no serious adverse effects were observed. Two participants reported experiencing headaches following the stimulation session, but these are not anticipated to be due to the gTMS stimulation as these effects were also observed after sham sessions.

Mild AD dementia is an early stage of Alzheimer's disease characterized by cognitive impairments, including deficits in episodic memory, executive function, language, and visuospatial abilities (Knopman et al., 2014). Disruptions in gamma oscillations, such as reduced power and synchronization, may contribute to these deficits (Liu et al., 2022; Kurudenkandy et al., 2014; Ahnaou et al., 2017; Reinhart et al., 2019; Nissim et al., 2023). Gamma neuromodulation, a non-invasive technique stimulating brain activity at approximately 40 Hz, showed promise in treating mild AD (Benussi et al., 2022; He et al., 2021; Chan et al., 2022; Traikapi et al., 2023; Benussi et al., 2021; Nissim et al., 2023). The present investigation demonstrated acute changes in brain oscillations resulting from a single low-intensity gTMS session. The findings reported herein provide compelling evidence of immediate gamma modulation following a single stimulation session, and highlight the potential of TMS, delivered at gamma frequencies and lower frequencies compared to traditional TMS, to enhance both local and distal oscillations within the brain while modulating gamma-band oscillations (Johnson et al., 2020; Alexander et al., 2019). By utilizing lower frequency gTMS protocols, safer long-term interventions can be ensured, which enable sustained positive effects on cognitive function. The lower intensity gTMS is more conducive to long-term repetitive use, which is more useful in improving cognitive and behavioral metrics.

The observed similarities in the theta frequency range suggest that gamma frequencies also facilitated theta-gamma coupling, a process implicated in various cognitive functions such as memory formation, attention, and information processing. Notably, disruptions in theta-gamma coupling have been observed in animal models and individuals with AD, underlining the potential relevance of gamma neuromodulation in AD physiopathology, for example as described in Goutagny R, et al., “Alterations in hippocampal network oscillations and theta-gamma coupling arise before AB overproduction in a mouse model of Alzheimer's disease,” European Journal of Neuroscience, Vol. 37, 2013: pp. 1896-902; and Goodman M S, et al., “Theta-Gamma Coupling and Working Memory in Alzheimer's Dementia and Mild Cognitive Impairment,” Frontiers in Aging Neuroscience, Vol. 10, No. 101, 2018, each of which is hereby incorporated by reference in their entirety.

The results highlighted a positive effect of a single session of low-intensity gTMS specifically targeting the precuneus region. Significant changes were observed in the power of gamma, theta, and alpha oscillations in patients with mild AD dementia. Although these physiological changes did not directly translate into improvements in cognition after a single session, they confirmed gamma synchronization was achieved through the application of a single low-intensity gTMS session, which is indicative of the potential for future, sustained improvements in cognition. Furthermore, the results suggested that this neuromodulation approach has a favorable safety profile, making it suitable for long-term interventions, which supports treatment for cognitive and/or behavioral improvement. The absence of adverse effects supports the potential viability of utilizing low-intensity gTMS in clinical settings and/or over longer periods of time.

### REFERENCES

Qiu C, et al. “Epidemiology of Alzheimer's disease: occurrence, determinants, and strategies toward intervention,” Dialogues in Clinical Neuroscience, Vol. 11, No. 2, 2009: pp. 111-28.
Benussi A, et al. “Increasing Brain Gamma Activity Improves Episodic Memory and Restores Cholinergic Dysfunction in Alzheimer's Disease,” Ann Neurol, Vol. 92, No. 2, 2022: pp. 322-34.
Liu C, et al. “Modulating Gamma Oscillations Promotes Brain Connectivity to Improve Cognitive Impairment,” Cerebral Cortex, Vol. 32, 2022: pp. 2644-56.
He Q, et al. “A feasibility trial of gamma sensory flicker for patients with prodromal Alzheimer's disease,” Alzheimers Dement (NY), Vol. 7, No. 1, 2021: e12178.
Buzsáki G & Wang X-J, “Mechanisms of Gamma Oscillations,” Annual Review of Neuroscience, Vol. 35, 2012: pp. 203-25.
Buzsáki G & Watson B O, “Brain rhythms and neural syntax: implications for efficient coding of cognitive content and neuropsychiatric disease,” Dialogues in Clinical Neuroscience, Vol. 14, No. 4, 2012: pp. 345-67.
Adaikkan C, et al. “Gamma Entrainment Binds Higher-Order Brain Regions and Offers Neuroprotection,” Neuron, Vol. 102, 2019: pp. 929-943, e8.
Etter G, et al. “Optogenetic gamma stimulation rescues memory impairments in an Alzheimer's disease mouse model,” Nat Commun, Vol. 10, No. 5322, 2019.
Chan D, et al. “Gamma frequency sensory stimulation in mild probable Alzheimer's dementia patients: Results of feasibility and pilot studies,” PLOS One, Vol. 17, No. 12, 2022: e0278412.
Traikapi A, et al. “Episodic memory effects of gamma frequency precuneus transcranial magnetic stimulation in Alzheimer's disease: A randomized multiple baseline study,” J Neuropsychol, Vol. 17, No. 2, 2023: pp. 279-301.
Sprugnoli G, et al. “Impact of multisession 40 Hz tACS on hippocampal perfusion in patients with Alzheimer's disease,” Alzheimers Res Ther, Vol. 13, No. 1, 2021:203.
Benussi A, et al. “Exposure to gamma tACS in Alzheimer's disease: A randomized, double-blind, sham-controlled, crossover, pilot study,” Brain Stimul, Vol. 14, No. 3, 2021: pp. 531-40.
Herwig U, et al. “Using the international 10-20 EEG system for positioning of transcranial magnetic stimulation,” Brain Topogr, Vol. 16, No. 2, 2003: pp. 95-9.
Gramfort A, et al. “MEG and EEG data analysis with MNE-Python,” Frontiers in Neuroscience, Vol. 7, No. 267, 2013:13 pages.
Harris C R, et al. “Array programming with NumPy,” Nature, Vol. 585, 2020: pp. 357-62.
Hunter J D. “Matplotlib: A 2D Graphics Environment,” Computing in Science & Engineering, Vol. 9, 2007: pp. 90-5.
Slepian D. “Prolate Spheroidal Wave Functions, Fourier Analysis, and Uncertainty—V: The Discrete Case,” Bell System Technical Journal, Vol. 57, 1978: pp. 1371-430.
Genovese C R, et al. “Thresholding of Statistical Maps in Functional Neuroimaging Using the False Discovery Rate,” NeuroImage, Vol. 15, No. 4, 2002: pp. 870-8.
Bean J. “Rey Auditory Verbal Learning Test, Rey AVLT,” In: Kreutzer J S, DeLuca J, Caplan B, editors. Encyclopedia of Clinical Neuropsychology, New York, NY: Springer; 2011, p. 2174-5.
Patterson J. “Verbal Fluency,” In: Kreutzer J S, DeLuca J, Caplan B, editors. Encyclopedia of Clinical Neuropsychology, New York, NY: Springer; 2011, p. 2603-6.
Knopman D S & Petersen R C. “Mild Cognitive Impairment and Mild Dementia: A Clinical Perspective,” Mayo Clin Proc, Vol. 89, No. 10, 2014: pp. 1452-9.
Kurudenkandy F R, et al. “Amyloid-B-Induced Action Potential Desynchronization and Degradation of Hippocampal Gamma Oscillations Is Prevented by Interference with Peptide Conformation Change and Aggregation,” J Neurosci, Vol. 34, 2014: pp. 11416-25.
Ahnaou A, et al. “Emergence of early alterations in network oscillations and functional connectivity in a tau seeding mouse model of Alzheimer's disease pathology,” Sci Rep, Vol. 7, 2017:14189.
Reinhart R M G & Nguyen J A, “Working memory revived in older adults by synchronizing rhythmic brain circuits,” Nat Neurosci, Vol. 22, 2019: pp. 820-7.
Nissim N R, et al. “The impact of gamma transcranial alternating current stimulation (tACS) on cognitive and memory processes in patients with mild cognitive impairment or Alzheimer's disease: A literature review,” Brain Stimulation, Vol. 16, 2023: pp. 748-55.
Johnson L, et al. “Dose-dependent effects of transcranial alternating current stimulation on spike timing in awake nonhuman primates. Science Advances, Vol. 6, 2020: eaaz2747.
Alexander M L, et al. “Double-blind, randomized pilot clinical trial targeting alpha oscillations with transcranial alternating current stimulation (tACS) for the treatment of major depressive disorder (MDD),” Transl Psychiatry, Vol. 9, No. 106, 2019: pp. 1-12.
Goutagny R, et al. “Alterations in hippocampal network oscillations and theta-gamma coupling arise before AB overproduction in a mouse model of Alzheimer's disease,” European Journal of Neuroscience, Vol. 37, 2013: pp. 1896-902.
Goodman M S, et al. “Theta-Gamma Coupling and Working Memory in Alzheimer's Dementia and Mild Cognitive Impairment,” Frontiers in Aging Neuroscience, Vol. 10, No. 101, 2018: pp. 1-10.

### Definitions

The following definitions are used in connection with the disclosure disclosed herein. Unless defined otherwise, all technical and scientific terms used herein have the same meaning as commonly understood to one of skill in the art to which this disclosure belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

In embodiments, as used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

### EQUIVALENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

What is claimed is:

1. A repetitive transcranial magnetic stimulation (rTMS/gTMS) method for treating, slowing, and/or preventing Alzheimer's disease (AD) and/or for treating, slowing, and/or preventing a conversion of mild cognitive impairment (MCI) to AD, comprising repetitively applying a magnetic pulse to the scalp of a subject in need thereof thereby stimulating neurons in the brain of the subject, wherein the magnetic pulse is applied:
a) repetitively over the subject's brain; and
b) at a frequency of less than about 50 Hz and an intensity of about 20,000 milligauss (20 gauss) to about 250,000 milligauss (250 gauss).

2. The method of claim 1, wherein the magnetic pulse is applied at a frequency of about 15 Hz to about 30 Hz, or about 30 Hz to about 40 Hz, optionally wherein the magnetic pulse is applied at a frequency of about 40 Hz.

3. The method of claim 1, wherein the magnetic pulse is applied at an intensity of 20,000 to about 25,000 milligauss, about 25,000 to about 30,000 milligauss, about 30,000 to about 35,000 milligauss, about 35,000 to about 40,000 milligauss, about 45,000 to about 50,000 milligauss, about 55,000 to about 60,000 milligauss, about 65,000 to about 70,000 milligauss, about 75,000 to about 80,000 milligauss, about 85,000 to about 90,000 milligauss, about 95,000 to about 100,000 milligauss, about 20,000 to about 100,000 milligauss, about 100,000 to about 120,000 milligauss, about 120,000 to about 140,000 milligauss, about 140,000 to about 160,000 milligauss, about 160,000 to about 180,000 milligauss, about 180,000 to about 200,000 milligauss, about 100,000 to about 200,000 milligauss, or about 200,000 to about 250,000 milligauss.

4. The method of claim 1, wherein the magnetic pulse generates an electric field of about 0.1 V/m² to about 10 V/m².

5. The method of claim 1, wherein the method is performed:
at least once daily, at least twice daily, at least thrice daily, or at least four times daily; and/or
for greater than about 15 minutes, for about 15 to about 60 minutes, optionally for about 30 minutes.

6. The method of claim 1, wherein the one or more magnetic pulses are applied at least about 100 to at least about 500 times.

7. The method of claim 1, wherein the one or more magnetic pulses last for a period of at least about 2 seconds, at least about 4 seconds, at least about 6 seconds, at least about 8 seconds, at least about 10 seconds, at least about 12 seconds, at least about 14 seconds, at least about 16 seconds, at least about 18 seconds, or at least about 20 seconds.

8. The method of claim 1, wherein the method is applied for at least about 2 months, for at least about 6 months, or for at least about 1 year or longer.

9. The method of claim 1, wherein the method is self-applied.

10. The method of claim 1, wherein the one or more magnetic pulses are applied using a device configured for conducting an electric current through one or more coils to generate a magnetic field, optionally wherein the device is suitable for home use, is portable, and/or is wearable, further optionally wherein the device is configured for use as a helmet or headband.

11. The method of claim 1, wherein the subject presents with one or more of:
MCI, amnestic type MCI, or non-amnestic type MCI;
preclinical Alzheimer's disease, mild Alzheimer's disease, or moderate Alzheimer's disease;
mild dementia; and/or
having at least one biomarker indicative of AD, selected from high amyloid beta (Aβ) in cerebrospinal fluid, high Tau in cerebrospinal fluid, and the presence of the ApoE4 allele.

12. The method of claim 1, further comprising measuring and/or assessing:
at least one biomarker indicative of AD, selected from high amyloid beta (Aβ) in cerebrospinal fluid, high Tau in cerebrospinal fluid, and the presence of the ApoE4 allele; and/or
one or more symptoms of the MCI, AD and/or dementia, wherein the measuring and/or assessing occurs prior applying the rTMS/gTMS and/or after applying the rTMS/gTMS.

13. The method of claim 12, wherein the measuring and/or assessing is performed using one or more methods, tests, and/or scales listed in Table 1.

14. The method of claim 1, wherein the method stimulates neurons from about 2 to about 3 cm from the skull.

15. The method of claim 1, wherein the method stimulates alterations in gamma, theta, alpha, and/or beta oscillation in the brain.

16. The method of claim 15, wherein the method stimulates alterations in gamma oscillation and/or theta oscillation in the brain.

17. The method of claim 1, wherein the one or more magnetic pulses substantially stimulates neurons in one or more of at least a portion of the cerebral cortex, left hemisphere of the brain, left prefrontal dorsolateral cortex, frontal lobe of the brain, and/or throughout the brain.

18. The method of claim 1, wherein the method improves cognitive traits of the subject, prevents diminution of cognitive traits of the subject, slows the subject's memory loss, and/or retains and/or increases memory capacity, memory function, and/or cognitive function in the subject.

19. The method of claim 18, wherein memory capacity, memory function, cognitive function, and/or memory loss is assessed using one or more of the Instruments for assessment of AD symptoms listed in Table 1.

20. The method of claim 1, wherein the method is substantially free of adverse effects, optionally selected from epileptic seizures, nausea, headache, vagal response, musculoskeletal pain, scalp petechial rash, scalp pain, edema, and bruising.
